SEQUENCE LISTING (12 sequences)

SEQ ID NO: 1 - DNA, 19 bp, synthetic construct
  misc_feature 1..19: note = Description of Artificial Sequence: Synthetic oligonucleotide
  source 1..19: mol_type = other DNA
aagtctctcagctggtaca

SEQ ID NO: 2 - DNA, 20 bp, synthetic construct
  misc_feature 1..20: note = Description of Artificial Sequence: Synthetic oligonucleotide
  source 1..20: mol_type = other DNA
gagccatgcttggcttacga

SEQ ID NO: 3 - protein, 20 aa, unidentified
  REGION 1..20: note = Description of Unknown: Nuclear localization sequence
AVKRPAATKKAGQAKKKKLD

SEQ ID NO: 4 - protein, 25 aa, unidentified
  REGION 1..25: note = Description of Unknown: Nuclear localization sequence
MSRRRKANPTKLSENAKKLAKEVEN

SEQ ID NO: 5 - protein, 9 aa, unidentified
  REGION 1..9: note = Description of Unknown: Nuclear localization sequence
PAAKRVKLD

SEQ ID NO: 6 - protein, 9 aa, unidentified
  REGION 1..9: note = Description of Unknown: Nuclear localization sequence
KLKIKRPVK

SEQ ID NO: 7 - protein, 7 aa, unidentified
  REGION 1..7: note = Description of Unknown: Nuclear localization sequence
PKKKRKV

SEQ ID NO: 8 - DNA, 23 bp, synthetic construct
  misc_feature 1..23: note = Description of Artificial Sequence: Synthetic oligonucleotide
  source 1..23: mol_type = other DNA
gagccatgcttggcttacgaggg

SEQ ID NO: 9 - DNA, 450 bp, synthetic construct
  misc_feature 1..450: note = Description of Artificial Sequence: Synthetic polynucleotide
  source 1..450: mol_type = other DNA
cgaccaacccatcaaactccccgcccccagcacttttatttctcctctttaggaagtacacttcagtatctttggcacagtgcatgagcacgactaaagtaaaacatcgcagaaaacatagctttagtctacccttcgtgtcctaaaaggaaaaccagtagcttcccaggccaccggaagggcaacacatgtcctctgcagtttctgcacacgggaaggtaaagacagagagaggacctactcctcaacacagaaacatttcaaaatctttcctcgcctgcaacccaagctgaagtcattctccccagaaataacaaaagttggaagagaagccggagacaggataggtgcaggaagcccacactttgagggcagcactcagacaccctctcctgtgtgcaggacgtgccgaatgttcaggtgcaatgagaatgagccatgcttggctta

SEQ ID NO: 10 - DNA, 3121 bp, synthetic construct
  misc_feature 1..3121: note = Description of Artificial Sequence: Synthetic polynucleotide
  source 1..3121: mol_type = other DNA
cgagggcaatctggcccatcaagtggccttcgcctctgggagtaacaaaaatgcacttcaaaatagcttctgtaatcaagctgcatgggtggagtactccccagctgactccaggaagttctctatccaaagctattcattaggccagagctgtgcaaataattagtcacccacttgctccataaccctccatgacagcccaggcattgagtccaggtgggaccatcaagccatgctctggtggctcatgcattatcatagaaatgggaggctttatttattttactaaaaagaacaaaaacaacagactgctgtcctttagacaataggatcacgtcatctgagccctctgtgccccaggtgacaagcccagccccaagttctctttcctcagcctccccacacatgttctggaggagatgggcccagcaggctgctctgaggcctggcatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtctcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggat

SEQ ID NO: 11 - DNA, 473 bp, synthetic construct
  misc_feature 1..473: note = Description of Artificial Sequence: Synthetic polynucleotide
  source 1..473: mol_type = other DNA
gagccatgcttggcttacgagggcgaccaacccatcaaactccccgcccccagcacttttatttctcctctttaggaagtacacttcagtatctttggcacagtgcatgagcacgactaaagtaaaacatcgcagaaaacatagctttagtctacccttcgtgtcctaaaaggaaaaccagtagcttcccaggccaccggaagggcaacacatgtcctctgcagtttctgcacacgggaaggtaaagacagagagaggacctactcctcaacacagaaacatttcaaaatctttcctcgcctgcaacccaagctgaagtcattctccccagaaataacaaaagttggaagagaagccggagacaggataggtgcaggaagcccacactttgagggcagcactcagacaccctctcctgtgtgcaggacgtgccgaatgttcaggtgcaatgagaatgagccatgcttggctta

SEQ ID NO: 12 - DNA, 3144 bp, synthetic construct
  misc_feature 1..3144: note = Description of Artificial Sequence: Synthetic polynucleotide
  source 1..3144: mol_type = other DNA
cgagggcaatctggcccatcaagtggccttcgcctctgggagtaacaaaaatgcacttcaaaatagcttctgtaatcaagctgcatgggtggagtactccccagctgactccaggaagttctctatccaaagctattcattaggccagagctgtgcaaataattagtcacccacttgctccataaccctccatgacagcccaggcattgagtccaggtgggaccatcaagccatgctctggtggctcatgcattatcatagaaatgggaggctttatttattttactaaaaagaacaaaaacaacagactgctgtcctttagacaataggatcacgtcatctgagccctctgtgccccaggtgacaagcccagccccaagttctctttcctcagcctccccacacatgttctggaggagatgggcccagcaggctgctctgaggcctggcccctcgtaagccaagcatggctcatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtctcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggat

(19) United States
(12) Patent Application Publication
(10) Pub. No.: US 2024/0240164 A1
(43) Pub. Date: Jul. 18, 2024

(54) NON-VIRAL HOMOLOGY MEDIATED END JOINING

(71) Applicant: Arsenal Biosciences, Inc., South San Francisco, CA (US)

(72) Inventors: Aaron Cooper, Berkeley, CA (US); Pei-Ken Hsu, San Francisco, CA (US); Robert Moot, San Francisco, CA (US); Annie Truong, San Carlos, CA (US); Thomas Gardner, San Francisco, CA (US); Brian Hsu, Foster City, CA (US)

(21) Appl. No.: 18/379,912

(22) Filed: Oct. 13, 2023

Related U.S. Application Data
(63) Continuation of application No. PCT/US2022/024703, filed on Apr. 13, 2022.
(60) Provisional application No. 63/174,468, filed on Apr. 13, 2021.

(51) Int. Cl. C12N 9/22; C12N 15/11; C12N 15/90

(52) U.S. Cl. CPC C12N 9/22; C12N 15/11; C12N 15/907; C12N 2310/20; C12N 2800/80

(57) ABSTRACT

Provided herein are compositions and methods for producing genetically engineered cells, such as T cells, with an insertion of a cassette at a designated genomic locus. Methods of using the genetically engineered cells for treating or preventing a disease in a subject are also provided.

### CROSS-REFERENCE TO RELATED APPLICATIONS

This application is a continuation of PCT/US2022/024703, filed on Apr. 13, 2022, which claims the benefit of U.S. provisional application No. 63/174,468 filed Apr. 13, 2021, which is hereby incorporated by reference in its entirety for all purposes.

### SEQUENCE LISTING

The instant application contains a Sequence Listing which has been submitted via Patent Center and is hereby incorporated by reference in its entirety. Said .xml copy, created on Dec. 27, 2023 is named ANB-206WOC1, and is 23,736 bytes in size.

### BACKGROUND OF THE DISCLOSURE

The application of clustered regularly interspaced short palindromic repeats (CRISPR) and CRISPR-associated (Cas) proteins has revolutionized molecular biology by making genome editing possible. CRISPR-mediated gene editing is a powerful and practical tool with potential for creating new scientific tools, correcting clinically relevant mutations, and engineering new cell-based immunotherapies. Viral delivery vectors and electroporation have emerged as two strategies for CRISPR-based editing in immune cells. However, the field of gene therapy has been fraught with complications of viral delivery vectors.

The ability to manipulate T cells, hematopoietic stem cells, and induced pluripotent stems cells provides both a scientific tool and a potentially therapeutic avenue to immune-engineering. Until the discovery of CRISPR-Cas gene editing in human cells, gene therapy directed towards the immune system relied almost exclusively upon viral vectors such as retroviruses and lentiviruses to insert a transgene by a (semi)random genomic integration. However, clinical trials have had unintended consequences, such as leukemia, due to insertion into proto-oncogenes and fatal systemic immune responses to the viral vector. Despite improvements in viral vectors to minimize genetic damage and immune responses, viral insertions (such as lentivirus used clinically to generate CAR-T cells) can result in gene over-expression, e.g., overexpression of genes that are not under endogenous regulation, restricting the clinical utility of this method.

Non-viral CRISPR-Cas gene editing has been described. However, editing efficiency and/or viability using such systems are generally low. For example, increased editing efficiency using current methods come at the cost of reduced cellular viability, and vice versa. Improved non-viral CRISPR-Cas gene editing methods are needed, such as methods having improved editing efficiency and viability.

### SUMMARY

Provided herein is a composition for modifying a target nucleic acid, comprising: (a) a targetable nuclease protein; and (b) a plasmid donor template comprising: (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein each of the first and the second CDL target sequences are capable of cleavage by the targetable nuclease protein or a complex comprising the targetable nuclease protein, and wherein the composition is formulated for non-viral delivery into a cell.

Also provided herein is A composition for modifying a target nucleic acid, comprising: (a) a CRISPR-CAS RNA-guided nuclease: (b) a donor guide RNA (gRNA); and (c) a plasmid donor template comprising: (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3′ of the CDL target sequences, and wherein the composition is formulated for non-viral delivery into a cell.

Also provided herein is A method for modifying a target nucleic acid of a cell, the method comprising: providing the cell, and introducing or having introduced into the cell a composition formulated for non-viral delivery, the composition comprising: (a) a targetable nuclease protein; and (b) a plasmid donor template comprising: (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein each of the first and the second CDL target sequences are capable of cleavage by the targetable nuclease protein or a complex comprising the targetable nuclease protein.

Also provided for herein is a method for modifying a target nucleic acid of a cell, the method comprising: providing the cell, and introducing or having introduced into the cell a composition formulated for non-viral delivery, the composition comprising: (a) a CRISPR-CAS RNA-guided nuclease: (b) a donor guide RNA (gRNA); and (c) a plasmid donor template comprising: (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3′ of the CDL target sequences.

Also provided herein is a method for modifying a genomic target sequence of a cell, the method comprising: providing the cell, and introducing or having introduced into the cell a composition formulated for non-viral delivery, the composition comprising: (a) a CRISPR-CAS RNA-guided nuclease: (b) a donor guide RNA (gRNA); and (c) a plasmid donor template comprising: (i) a homology directed repair (HDR) template comprising a nucleic acid for insertion flanked by homology arms: (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3′ of the CDL target sequences, wherein the donor gRNA comprises at least 17 nucleotides that are complementary to the genomic target sequence of the cell, and wherein the homology arms are complementary to nucleic acid sequences flanking the genomic target sequence of the cell, wherein the nucleic acid for insertion is configured for insertion into the genomic target sequence of the cell.

In some aspects, the targetable nuclease protein is an RNA-guided nuclease. In some aspects, the composition further comprises an RNA comprising at least 17 nucleotides that are complementary to the CDL target sequence. In some aspects, the RNA-guided nuclease is a Cas protein.

In some aspects, the composition further comprises a donor guide RNA (gRNA) configured to form the complex comprising the targetable nuclease protein, and wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3′ of the CDL target sequences. In some aspects, the composition further comprises a first donor guide RNA (gRNA) comprising at least 17 nucleotides that are complementary to the first CDL target sequence, a second donor gRNA comprising at least 17 nucleotides that are complementary to the second CDL target sequence, wherein each donor gRNA is configured to form a distinct complex comprising the targetable nuclease protein, and wherein each of the first and the second CDL target sequences are operably linked to a 3-base pair protospacer adjacent motif (PAM) located 3′ of the CDL target sequences. In some aspects, one or more of the donor gRNAs comprises at least 17 nucleotides that are complementary to a genomic target sequence of the cell. In some aspects, the HDR template comprises homology arms that are complementary to nucleic acid sequences flanking the genomic target sequence of the cell. In some aspects, the homology arms are each independently selected from a length of at least 400 bp, at least 500 bp, at least 600 bp, at least 700 bp, at least 800 bp, at least 900 bp, 1000 bp, at least 1100 bp, at least 1200 bp, at least 1300 bp, 1400 bp, at least 1500 bp, at least 1600 bp, at least 1700 bp, at least 1800 bp, at least 1900 bp, or at least 2000 bp in length.

In some aspects: (a) the targetable nuclease comprises an RNA-guided nuclease, wherein the RNA-guided nuclease comprises CRISPR-CAS: (b) the composition further comprises a donor guide RNA (gRNA) configured to form the complex comprising the targetable nuclease protein; and (c) wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3′ of the CDL target sequences.

In some aspects, the composition further comprises a second targetable nuclease protein, wherein the second targetable nuclease protein or a complex comprising the second targetable nuclease protein is capable of cleaving a genomic target sequence of the cell. In some aspects, the second targetable nuclease protein is an RNA-guided nuclease. In some aspects, the composition further comprises a second RNA comprising at least 17 nucleotides that are complementary to the genomic target sequence. In some aspects, the RNA-guided nuclease is a Cas protein. In some aspects, the composition further comprises a target guide RNA (gRNA) configured to form the complex comprising the second targetable nuclease protein, and wherein (1) the target gRNA comprises at least 17 nucleotides that are complementary to the genomic target sequence, and (2) the genomic target sequence is operably linked to a 3-base pair protospacer adjacent motif (PAM) located 3′ of the genomic target sequence.

In some aspects, the first CDL target sequence, the second CDL target sequence, and the genomic target sequence comprise the same nucleic acid sequence.

In some aspects, the genomic target sequence comprises a safe-harbor nucleic acid sequence. In some aspects, the safe-harbor nucleic acid sequence comprises the nucleic acid sequence GAGCCATGCTTGGCTTACGA (SEQ ID NO; 2). In some aspects, one, both, or neither of the PAM sequences are encoded between the CDL target sequences and the HDR template.

In some aspects, the targetable nuclease and each of the gRNAs are in a molar ratio of between 1:10 and 2:1, respectively. In some aspects, the targetable nuclease and the donor template are in a molar ratio of between 10:1 and 1000:1, respectively.

In some aspects, the targetable nuclease protein and/or the second targetable nuclease protein comprises a transcription activator-like (TAL) effector DNA-binding protein and a nuclease.

In some aspects, the targetable nuclease protein and/or the second targetable nuclease protein comprises a zinc finger DNA-binding protein and a nuclease.

In some aspects, the targetable nuclease protein and/or the second targetable nuclease protein is fused to a nuclear localization signal (NLS) sequence.

In some aspects, the targetable nuclease protein and/or the second targetable nuclease protein is a Cas9 protein.

Also provided herein is a method for modifying the target nucleic acid in the cell, comprising introducing or having non-virally introduced into the cell any of the compositions described herein, wherein the HDR template is integrated into the target nucleic acid. In some aspects, the introducing comprises electroporation. In some aspects, the cell is a primary cell. In some aspects, the primary cell is a primary T cell.

Also provided for herein is a ribonucleoprotein complex for modifying a target nucleic acid, comprising any of the compositions described herein.

Also provided for herein is a ribonucleoprotein complex for modifying a target nucleic acid, (a) a CRISPR-CAS RNA-guided nuclease; and (b) a donor guide RNA (gRNA), wherein the donor gRNA comprises at least 17 nucleotides that are complementary to a co-delivery linearization (CDL) target sequence, and wherein the composition is formulated for non-viral delivery into a cell. In some aspects, the composition further comprises a plasmid donor template comprising: (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3′ of the CDL target sequences.

Also provided for herein is a method for modifying the target nucleic acid in the cell, comprising introducing into the cell any of the ribonucleoprotein complex described herein. In some aspects, the introducing comprises electroporation. In some aspects, the cell is a primary cell. In some aspects, the primary cell is a primary T cell. In some aspects, the method is performed in vivo, in vitro, or ex vivo.

Also provided for herein is a method of forming a ribonucleoprotein (RNP) complex, comprising incubating or having incubated (a) a CRISPR-CAS RNA-guided nuclease; and (b) a donor guide RNA (gRNA), wherein the donor gRNA comprises at least 17 nucleotides that are complementary to a co-delivery linearization (CDL) target sequence. In some aspects, the Cas protein and the gRNA are incubated together at 37° C. for at least 17 minutes. In some aspects, the molar ratio of gRNA; Cas protein is between 0.25:1 and 4:1. In some aspects, the RNP complex has a size that is less than 100 nm. In some aspects, the RNP complex has a size that is between 20 nm and 90 nm.

Also provided herein is a composition comprising a plasmid donor template comprising: (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein each of the first and the second CDL target sequences are capable of cleavage by a targetable nuclease protein or a complex comprising the targetable nuclease protein. In some aspects, the composition further comprises a donor guide RNA (gRNA) configured to form the complex comprising the targetable nuclease protein. In some aspects, the composition comprises the targetable nuclease protein.

In some aspects, the donor template comprises from 5′ to 3′ the sequence: Pla-N1-P2b-H-P3c-N2-P4d, and wherein: (1) P1, P2, P3, and P4 are PAM sequences: (2) N1 is the first CDL target sequence and N2 is the second CDL target sequence: (3) H is the HDR template; (4) a is 0 and b is 1, or a is 1 and b is 0 and (5) c is 0 and d is 1; or c is 1 and dis 0).

In certain aspects, described herein is a method for modifying a target nucleic acid of a cell, the method comprising: providing the cell, and introducing or having introduced into the cell a composition formulated for non-viral delivery, the composition comprising: (a) a targetable nuclease protein; and (b) a plasmid donor template comprising: (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3″ of the HDR template, and wherein each of the first and the second CDL target sequences are capable of cleavage by the targetable nuclease protein or a complex comprising the targetable nuclease protein. In certain aspects, described herein is a method for modifying a target nucleic acid of a cell, the method comprising: providing the cell, and introducing or having introduced into the cell a composition formulated for non-viral delivery, the composition comprising: (a) a CRISPR-CAS RNA-guided nuclease: (b) a donor guide RNA (gRNA); and (c) a plasmid donor template comprising: (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3″ of the CDL target sequences. In certain aspects, described herein is a method for modifying a genomic target sequence of a cell, the method comprising: providing the cell, and introducing or having introduced into the cell a composition formulated for non-viral delivery, the composition comprising: (a) a CRISPR-CAS RNA-guided nuclease: (b) a donor guide RNA (gRNA); and (c) a plasmid donor template comprising: (i) a homology directed repair (HDR) template comprising a nucleic acid for insertion flanked by homology arms; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3″ of the CDL target sequences wherein the donor gRNA comprises at least 17 nucleotides that are complementary to the genomic target sequence of the cell, and wherein the homology arms are complementary to nucleic acid sequences flanking the genomic target sequence of the cell, wherein the nucleic acid for insertion is configured for insertion into the genomic target sequence of the cell. In certain embodiments, the cell is a human cell. In certain embodiments, the cell is an immune cell. In certain embodiments, the immune cell is a T cell. In certain embodiments, the T cell is a primary T cell. In certain embodiments, the introducing the composition formulated for non-viral delivery comprises electroporation. In certain embodiments, the amount of donor template is at least about 80, 10-120, 10, 20, 30, 40, 50, 60, 70, 90, 100, 110, or 120 μg. In certain embodiments, the number of cells for an individual electroporation reaction is at least about 5, 1-10, 1, 2, 3, 4, 6, 7, 8, 9, or 10 e7. In certain embodiments, the total number of cells provided is at least greater than 10 e7 and greater than one electroporation reaction is performed. In certain embodiments, the total volume of cell suspension is about 1 mL. In certain embodiments, the method results in increased template insertion in the genomic target sequence of the cells relative to an otherwise identical control composition but lacking the CDL target sequences, optionally wherein the template insertion increases by at least about 1-5, 1, 2, 3, 4, or 5 fold relative to the control.

### Definitions

As used in this specification and the appended claims, the singular forms “a,” “an.” and “the” include plural reference unless the context clearly dictates otherwise.

As used herein, the “CRISPR-Cas” system refers to a class of bacterial systems for defense against foreign nucleic acid. CRISPR-Cas systems are found in a wide range of eubacterial and archaeal organisms. CRISPR-Cas systems include type I, II, and III sub-types. Wild-type type II CRISPR-Cas systems utilize an RNA-mediated nuclease, for example, Cas9 protein, in complex with guide and activating RNA (e.g., single-guide RNA or sgRNA) to recognize and cleave foreign nucleic acids, e.g., foreign nucleic acids including natural or modified nucleotides.

As used herein, the term “targetable nuclease” refers to a protein that can recognize a sequence of a cognate nucleic acid sequence (e.g., a target gene within a genome and/or a CDL target sequence) and bind to the cognate nucleic acid sequence. In some embodiments, the targetable nuclease can modify cognate nucleic acid sequence. In some embodiments, a targetable nuclease can be an RNA-guided nuclease, e.g., a Cas protein. In other embodiments, a targetable nuclease can be a fusion protein that includes a protein that can bind to a cognate nucleic acid sequence (e.g., a transcription activator-like (TAL) effector DNA-binding protein or a zinc finger DNA-binding protein) and a protein that can modify a cognate nucleic acid sequence (e.g., a nuclease, a transcription activator or repressor). In some embodiments, a targetable nuclease has nuclease activity. In other embodiments, a targetable nuclease does not have nuclease activity. In some embodiments, a targetable nuclease can modify a cognate nucleic acid sequence by cleaving the target nucleic acid. The cleaved target nucleic acid can then undergo homologous recombination with a nearby a homology directed repair (HDR) template, such as through homology directed repair or homology mediated end joining (HMEJ). In other embodiments, a targetable nuclease (e.g., a targetable nuclease without any nuclease activity) can regulate the expression of cognate nucleic acid sequence. For example, a targetable nuclease can be a fusion protein containing a TAL effector DNA-binding protein and a transcription activator.

As used herein, the term “plasmid donor template” refers to a polynucleotide that includes a homology directed repair (HDR) template and CDL target sequences. An HDR template can include a 5′ homology arm, a nucleotide insert (e.g., an exogenous sequence and/or a sequence that encodes a heterologous protein or fragment thereof), and a 3′ homology arm (for example, see FIG. 1). As described further herein, pre-incubation of the RNP complex containing a targetable nuclease (e.g., a Cas protein) and donor gRNA and a plasmid donor template prior to electroporation improves in knock-in yield, such as through improved knock-in efficiency and/or reduced cytotoxicity.

As used herein, the term “co-delivery linearization (CDL) target sequence” refers to a nucleotide sequence that is recognized and bound by a targetable nuclease. In some embodiments, a targetable nuclease, e.g., a transcription activator-like (TAL) effector DNA-binding protein or zinc finger DNA-binding protein, can directly recognize and bind a CDL target sequence. In other embodiments, a targetable nuclease, e.g., an RNA-guided nuclease, can indirectly recognize and bind a CDL target sequence via a donor gRNA. A RNA-guided nuclease binds to the donor gRNA, while the donor gRNA hybridizes to a CDL target sequence. In some embodiments, a CDL target sequence is a portion of genomic target nucleic acid.

As used herein, the “RNA-guided nuclease” refers to a nuclease that binds to a guide RNA (gRNA) and utilizes the gRNA to selectively bind regions within a DNA polynucleotide. In general, an RNA-guided nuclease can selectively bind nearly any sequence within a DNA polynucleotide that is complementary to the gRNA. In some embodiments, a RNA-guided nuclease has nuclease activity and can cleave the linkage (e.g., phosphodiester bonds) between nucleotides in the DNA polynucleotide. In other embodiments, a RNA-guided nuclease does not have nuclease activity and can be used to selectively bind and/or localize other proteins (e.g., transcriptional activator or repressors) that are fused to the RNA-guided nuclease to the region of interest within the DNA polynucleotide.

As used herein, the term “guide RNA” or “gRNA” refers to a DNA-targeting RNA that can guide an RNA-guided nuclease (e.g., a Cas protein) to a cognate nucleic acid sequence by hybridizing to the cognate nucleic acid sequence. In some embodiments, a guide RNA can be a single-guide RNA (sgRNA), which contains (1) a guide sequence (e.g., crRNA equivalent portion of the single-guide RNA) that guides a RNA-guided nuclease to a cognate nucleic acid sequence and (2) a scaffold sequence (e.g., tracrRNA equivalent portion of the single-guide RNA) that interacts with the RNA-guided nuclease. In other embodiments, a guide RNA can contain two components. (1) a guide sequence (e.g., crRNA equivalent portion of the single-guide RNA) that guides a RNA-guided nuclease to cognate nucleic acid sequence and (2) a scaffold sequence (e.g., tracrRNA equivalent portion of the single-guide RNA) that interacts with the RNA-guided nuclease. A portion of the guide sequence can hybridize to a portion of the scaffold sequence to form the two-component guide RNA.

As used herein, the term “target guide RNA” or “target gRNA” refers to a gRNA that can hybridize to a cognate nucleic acid sequence to be modified, e.g., at a location in a DNA polynucleotide where integration of an HDR template is desired, such as the genome of a T cell and/or safe-harbor genomic locations.

As used herein, the term “donor guide RNA” or “donor gRNA” refers to a gRNA that can hybridize a CDL target sequence within a plasmid donor template. In some embodiments, a CDL target sequence can be complementary (e.g., partially complementary or completely complementary) to an equal length portion of the sequence of a donor gRNA.

As used herein, the term “single-guide RNA” or “sgRNA” refers to a DNA-targeting RNA including (1) a guide sequence (e.g., crRNA equivalent portion of the single-guide RNA) that targets a Cas protein to a cognate nucleic acid sequence and (2) a scaffold sequence (e.g., tracrRNA equivalent portion of the single-guide RNA) that interacts with a Cas protein.

As used herein, the term “complementary” or “complementarity” refers to the capacity for base pairing between nucleobases, nucleosides, or nucleotides, as well as the capacity for base pairing between one polynucleotide to another polynucleotide. In some embodiments, one polynucleotide can have “complete complementarity.” or be “completely complementary,” to another polynucleotide, which means that when the two polynucleotides are optionally aligned, each nucleotide in one polynucleotide can engage in Watson-Crick base pairing with its corresponding nucleotide in the other polynucleotide. In other embodiments, one polynucleotide can have “partial complementarity.” or be “partially complementary.” to another polynucleotide, which means that when the two polynucleotides are optionally aligned, at least 60% (e.g., 65%, 70%, 75%, 80%, 85%, 90%, 95%, or 97%) but less than 100% of the nucleotides in one polynucleotide can engage in Watson-Crick base pairing with their corresponding nucleotides in the other polynucleotide. In other words, there is at least one (e.g., one, two, three, four, five, six, seven, eight, nine, or ten) mismatched nucleotide base pair when the two polynucleotides are hybridized. Pairs of nucleotides that engage in Watson-Crick base pairing includes, e.g., adenine and thymine, cytosine and guanine, and adenine and uracil, which all pair through the formation of hydrogen bonds. Examples of mismatched bases include a guanine and uracil, guanine and thymine, and adenine and cytosine pairing.

As used herein, the term “Cas protein” refers to a Clustered Regularly Interspaced Short Palindromic Repeats-associated protein or nuclease. A Cas protein can be a wild-type Cas protein or a Cas protein variant. Cas9 protein is an example of a Cas protein that belongs in the type II CRISPR-Cas system (e.g., Rath et al., Biochimie 117:119, 2015). Other examples of Cas proteins are described in detail further herein. A naturally-occurring type II Cas protein generally requires both a crRNA and a tracrRNA for site-specific DNA recognition and cleavage. The crRNA associates, through a region of partial complementarity, with the tracrRNA to guide the Cas protein to a region homologous to the crRNA in the target DNA called a “protospacer”. A naturally-occurring type II Cas protein cleaves DNA to generate blunt ends at the double-strand break at sites specified by a guide sequence contained within a crRNA transcript. In some embodiments of the compositions and methods described herein, a Cas protein associates with a target gRNA or a donor gRNA to form a ribonucleoprotein (RNP) complex. In some embodiments of the compositions and methods described herein, the Cas protein has nuclease activity. In other embodiments, the Cas protein does not have nuclease activity.

As used herein, the term “Cas protein variant” refers to a Cas protein that has at least one amino acid substitution (e.g., one, two, three, four, five, six, seven, eight, nine, ten, or more amino acid substitutions) relative to the sequence of a wild-type Cas protein and/or is a truncated version or fragment of a wild-type Cas protein. In some embodiments, a Cas protein variant has at least 75% sequence identity (e.g., at least 75%, 80%, 85%, 90%, 91%, 92%, 93%, 94% 95%, 96%, 97%, 98%, 99%, or 100% sequence identity) to the sequence of a wild-type Cas protein. In some embodiments, a Cas protein variant is a fragment of a wild-type Cas protein and has at least one amino acid substitution relative to the sequence of the wild-type Cas protein. A Cas protein variant can be a Cas9 protein variant. In some embodiments, a Cas protein variant has nuclease activity. In other embodiments, a Cas protein variant does not have nuclease activity.

As used herein, the term “ribonucleoprotein complex” or “RNP complex” refers to a complex comprising a Cas protein or variant (e.g., a Cas9 protein or variant) and a gRNA.

As used herein, the term “modifying” in the context of modifying a target nucleic acid in the genome of a cell refers to inducing a change (e.g., cleavage) in the target nucleic acid. In some embodiments, the change can be a structural change in the sequence of the target nucleic acid. For example, the modifying can take the form of inserting a nucleotide sequence into the target nucleic acid. For example, an exogenous nucleotide sequence can be inserted into the target nucleic acid. The target nucleic acid can also be excised and replaced with an exogenous nucleotide sequence. In another example, the modifying can take the form of cleaving the target nucleic acid without inserting a nucleotide sequence into the target nucleic acid. For example, the target nucleic acid can be cleared and excised. Such modifying can be performed, for example, by inducing a double stranded break within the target nucleic acid, or a pair of single stranded nicks on opposite strands and flanking the target nucleic acid. Methods for inducing single or double stranded breaks at or within a target nucleic acid include the use of a targetable nuclease (e.g., a Cas protein) as described herein directed to the target nucleic acid. In other embodiments, modifying a target nucleic acid includes targeting another protein to the target nucleic acid and does not include cleaving the target nucleic acid.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a conceptual drawing illustrating an exemplary mechanism of the CDL (Co-Delivery Linearization) plasmid design for mediating genomic insertion of a cassette. As shown on the right, adding the CDL sequence (a Cas9 target sequence identical to the Cas9 genomic target for HDR integration) and a 3-base pair protospacer adjacent motif (PAM) outside of the homology arms flanking the cassette allows the Cas9-sgRNA ribonucleoprotein (RNP)-mediated endonuclease activity to release the cassette plus homology arms from the plasmid, in addition to the Cas9-sgRNA RNP-mediated cut in the genome. On the contrary, with standard plasmid, shown on the left, Cas9-sgRNA RNP only cuts the genomic locus but not the plasmid.

FIG. 2A, 2B, and 2C are flow cytometry dot plots and graphs showing knock-in (KI) efficiency and KI cell yield of T-cells electroporated with a CRISPR RNP and homology-directed repair (HDR) templates of either standard plasmids or CDL plasmids. FIG. 2A shows a series of flow cytometry dot plots of T-cells electroporated with a CRISPR RNP and different doses (increasing from left to right, ranging between 0-100 mg/L) of homology-directed repair (HDR) templates of either standard plasmids (top panels) or CDL plasmids (bottom panels) encoding a MYC-tagged surface protein. FIG. 2B is a graph showing the KI % with varying doses of either standard plasmids or CDL plasmids. FIG. 2C is a graph showing the KI+ viable (live/dead stain: ThermoFisher) cell yield per 1 million starting cells with varying doses of either standard plasmids or CDL plasmids.

FIG. 3 is a graph showing the CD8/CD4 ratio with varying doses of either standard plasmids or CDL plasmids.

FIG. 4 is a graph showing transgene expression of T cells electroporated with 3 plasmids containing the CDL sequence GAGCCATGCTTGGCTTACGA (SEQ ID NO:2) and 3 plasmids containing non-CDL sequences on day 6 post-electroporation.

### DETAILED DESCRIPTION OF THE DISCLOSURE

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art: therefore, information well known to the skilled artisan is not necessarily included.

### I. Introduction

Virus-modified T cells are approved for cancer immunotherapy, but more versatile and precise genome modifications are needed for a wider range of adoptive cellular therapies (Yin et al., Nat Rev Clin Oncol, 16(5):281-295, 2019; Dunbar et al., Science 359:6372, 2018; Cornu et al., Nat Med 23:415-423, 2017; and David and Doherty, Toxicol Sci 155:315-325, 2017). The CRISPR (Clustered Regularly Interspaced Short Palindromic Repeats)-Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “single-guide RNA” or “sgRNA”), and the crRNA equivalent portion of the sgRNA can be engineered to guide the Cas (e.g., Cas9) nuclease to target any desired sequence (see, e.g., Jinek et al. (2012) Science 337:816-821; Jinek et al. (2013) eLife 2; e00471; Segal (2013) eLife 2; e00563). Thus, the CRISPR-Cas system can be engineered to create a double-strand break at a desired target in a genome of a cell, and harness the cell's endogenous mechanisms to repair the induced break by homology-directed repair (HDR) or nonhomologous end-joining (NHEJ).

As described herein, the inventors discovered sequences targeted by nucleases, when added at the ends of the homology directed repair (HDR) template, can enhance target nucleic acid modification efficiency. Non-viral strategies, such as electroporation, to deliver the CRISPR-Cas system into the cell for genetic modification avoid many complications associated with viral delivery, such as fatal systemic immune responses to viral vectors, viral delivery inefficiency, and viral insertion-related gene overexpression. However, in some cases, the need for large amounts of HDR template and dose-dependent cytotoxicity can occur when non-viral strategies for CRISPR-Cas system delivery are employed.

### II. Compositions

The disclosure provides compositions and methods for modifying a target nucleic acid that include: (a) a targetable nuclease protein; and (b) a plasmid donor template comprising: (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein each of the first and the second CDL target sequences are capable of cleavage by the targetable nuclease protein or a complex comprising the targetable nuclease protein, and wherein the composition is formulated for non-viral delivery into a cell. As described in detail further herein, in some embodiments, when the targetable nuclease is a transcription activator-like (TAL) effector, the TAL effector can directly recognize and bind to the CDL target sequence. In some embodiments, when the targetable nuclease is a zinc finger, the zinc finger can directly recognize and bind to the CDL target sequence. In other embodiments, when the targetable nuclease is an RNA-guided nuclease (e.g., a Cas protein), the RNA-guided nuclease can indirectly bind to a CDL target sequence via a donor gRNA, which can hybridize to the CDL target sequence. Without being bound by any theory, the targetable nuclease serves to cleave the CDL target sequence thus excising the HDR template from the plasmid donor template allowing the HDR template to participate in homology-mediated end joining (HMEJ). Knock-in efficiency can be maintained, or even increased, using lower amounts of plasmid donor template via the HMEJ-directed process described herein, as well as can result in reduced DNA-induced cytotoxicity. Thus, the CDL target sequences can improve the insertion efficiency of the HDR template into target cells, while also reducing toxicity, increasing overall yield of edited cells.

### III. CRISPR/Cas

In some embodiments of the compositions and methods described herein, the targetable nuclease is a RNA-guided nuclease, and the donor template further comprises protospacer adjacent motifs (PAMs) immediately adjacent to CDL target sequences. The composition can also further comprise a target guide RNA (gRNA) that is complementary to a target nucleic acid to be modified, e.g., a genomic target sequence, such as a desired site for transgene insertion in a cell (e.g., a T cell). The target gRNA can form a first RNP complex with the first RNA-guided nuclease and guide the first RNA-guided nuclease (e.g., Cas protein) to the target nucleic acid. In some embodiments, a portion of the target gRNA (e.g., a portion of the target gRNA that is at least 17 nucleotides (e.g., 17, 18, 19, 20, 21, 22, 23, 24, or 25 nucleotides) is complementary to the target nucleic acid.

The composition can also further comprise a donor gRNA that is complementary to the CDL target sequence. The donor gRNA can form a second RNP with the second RNA-guided nuclease. The CDL target sequence in the donor template can hybridize to the donor gRNA or a portion thereof. Therefore, the complex containing the second RNA-guided nuclease, the donor gRNA, and the donor template can facilitate excision of an HDR template and, without wishing to be bound by theory, promote homologous recombination to occur at the integration site in the target nucleic acid cleaved by the target gRNA. In some embodiments, the sequences of the target gRNA and the donor gRNA are the same. In some embodiments, the sequences of the target gRNA and the donor gRNA are different. In some embodiments, the first and second RNA-guided nucleases are the same. In other embodiments, the first and second RNA-guided nucleases are different.

The composition can contain a targetable nuclease and a target gRNA and/or a donor gRNA in a molar ratio of between 1:10 and 2:1 (e.g., between 1:5 and 2:1, between 2:5 and 2:1, between 3:5 and 2:1, between 4:5 and 2:1, between 1:1 and 2:1, between 1:10 and 1:1, between 1:10 and 4:5, between 1:10 and 3:5, between 1:10 and 2:5, or between 1:10 and 1:5), respectively. The composition can contain a targetable nuclease and a plasmid donor template in a molar ratio of between 10:1 and 1000:1 (e.g., between 50:1 and 1000:1, between 100:1 and 1000:1, between 200:1 and 1000:1, between 300:1 and 1000:1, between 400:1 and 1000:1, between 500:1 and 1000:1, between 600:1 and 1000:1, between 700:1 and 1000:1, between 800:1 and 1000:1, between 900:1 and 1000:1, between 10:1 and 900:1, between 10:1 and 800:1, between 10:1 and 700:1, between 10:1 and 600:1, between 10:1 and 500:1, between 10:1 and 400:1, between 10:1 and 300:1, between 10:1 and 200:1, between 10:1 and 100:1, or between 10:1 and 50:1), respectively.

A RNA-guided nuclease can also be fused with a localization peptide or protein. For example, the RNA-guided nuclease can be fused with one or more nuclear localization signal (NLS) sequences, which can direct the nuclease and the RNP complexes it forms to the nucleus to modify the target nucleic acid. Examples of NLS sequences are known in the art, e.g., as described in Lange et al., J Biol Chem. 282(8):5101-5, 2007, and also include, but are not limited to, AVKRPAATKKAGQAKKKKLD (SEQ ID NO; 3), MSRRRKANPTKLSENAKKLAKEVEN (SEQ ID NO; 4), PAAKRVKLD (SEQ ID NO; 5), KLKIKRPVK (SEQ ID NO; 6), and PKKKRKV (SEQ ID NO; 7). Examples of other peptide or proteins that can be used to a RNA-guided nuclease, such as cell-penetrating peptides and cell-targeting peptides are available in the art and described, e.g., Vivès et al., Biochim Biophys Acta. 1786(2); 126-38, 2008.

### IV. Single-Guide RNAs

A Cas protein may be guided to its respective cognate DNA to be cleaved (e.g., a CDL target sequence and/or genomic target sequence) by a single-guide RNA (sgRNA). An sgRNA is a version of the naturally occurring two-piece guide RNA (crRNA and tracrRNA) engineered into a single, continuous sequence. An sgRNA may contain a guide sequence (e.g., the crRNA equivalent portion of the sgRNA) that targets the Cas protein to the cognate nucleic acid sequence and a scaffold sequence that interacts with the Cas protein (e.g., the tracrRNAs equivalent portion of the sgRNA). An sgRNA may be selected using a software. As a non-limiting example, considerations for selecting an sgRNA can include, e.g., the PAM sequence for the Cas9 protein to be used, and strategies for minimizing off-target modifications. Tools, such as NUPACKR; and the CRISPR Design Tool, can provide sequences for preparing the sgRNA, for assessing target modification efficiency, and/or assessing cleavage at off-target sites.

### Guide Sequence

The guide sequence in the sgRNA may be complementary to a specific sequence within a cognate nucleic acid sequence (e.g., a CDL target sequence and/or genomic target sequence). The 3′ end of a cognate nucleic acid sequence can be followed by a PAM sequence. A guide sequence is generally complementary to approximately 20 nucleotides upstream of the PAM sequence. In general, a Cas9 protein or a variant thereof cleaves about three nucleotides upstream of a PAM sequence. The guide sequence in the sgRNA can be complementary to either strand of a cognate nucleic acid sequence.

In some embodiments, the guide sequence of an sgRNA may comprise about 10 to about 2000 nucleic acids, for example, about 10 to about 100 nucleic acids, about 10 to about 500 nucleic acids, about 10 to about 1000 nucleic acids, about 10 to about 1500 nucleic acids, about 10) to about 2000 nucleic acids, about 50 to about 100 nucleic acids, about 50 to about 500 nucleic acids, about 50) to about 1000 nucleic acids, about 50 to about 1500 nucleic acids, about 50) to about 2000 nucleic acids, about 100 to about 500 nucleic acids, about 100 to about 1000 nucleic acids, about 100 to about 1500 nucleic acids, about 100 to about 2000 nucleic acids, about 500) to about 1000 nucleic acids, about 500 to about 1500 nucleic acids, about 500 to about 2000 nucleic acids, about 1000 to about 1500 nucleic acids, about 1000 to about 2000 nucleic acids, or about 1500 to about 2000 nucleic acids at the 5′ end of the sgRNA that can direct the Cas protein to a cognate nucleic acid sequence using RNA-DNA complementarity base pairing. In some embodiments, the guide sequence of an sgRNA comprises about 100 nucleic acids at the 5′ end of the sgRNA that can direct the Cas protein to a cognate nucleic acid sequence site using RNA-DNA complementarity base pairing. In some embodiments, the guide sequence comprises 20 nucleic acids at the 5′ end of the sgRNA that can direct the Cas protein to a cognate nucleic acid sequence (e.g., a CDL target sequence and/or genomic target sequence) site using RNA-DNA complementarity base pairing. In other embodiments, the guide sequence comprises less than 20, e.g., 19, 18, 17 or less, nucleic acids that are complementary to a cognate nucleic acid sequence. In some instances, the guide sequence in the sgRNA contains at least one nucleic acid mismatch in the complementarity region of a cognate nucleic acid sequence. In some instances, the guide sequence contains about 1 to about 10 nucleic acid mismatches in the complementarity region of a cognate nucleic acid sequence.

### Scaffold Sequence

The scaffold sequence in the sgRNA may serve as a protein-binding sequence that interacts with the Cas protein or a variant thereof. In some embodiments, the scaffold sequence in the sgRNA can comprise two complementary stretches of nucleotides that hybridize to one another to form a double-stranded RNA duplex (dsRNA duplex). The scaffold sequence may have structures such as lower stem, bulge, upper stem, nexus, and/or hairpin. In some embodiments, the scaffold sequence in the sgRNA can be between about 90 nucleic acids to about 120 nucleic acids, e.g., about 90 nucleic acids to about 115 nucleic acids, about 90 nucleic acids to about 110 nucleic acids, about 90 nucleic acids to about 105 nucleic acids, about 90 nucleic acids to about 100 nucleic acids, about 90 nucleic acids to about 95 nucleic acids, about 95 nucleic acids to about 120 nucleic acids, about 100 nucleic acids to about 120 nucleic acids, about 105 nucleic acids to about 120 nucleic acids, about 110 nucleic acids to about 120 nucleic acids, or about 115 nucleic acids to about 120 nucleic acids.

V. Guide RNA (gRNA)

Guide RNAs (gRNAs), including target gRNAs and donor gRNAs described herein, in general refer to a DNA-targeting RNA containing (1) a guide sequence that is complementary to a cognate nucleic acid sequence (e.g., a CDL target sequence and/or genomic target sequence) and guides the RNA-guided nuclease to the cognate nucleic acid sequence and (2) a scaffold sequence that interacts and binds with the RNA-guided nuclease. In some embodiments of the disclosure, a target gRNA and a donor gRNA have the same sequence. In some embodiments of the disclosure, a target gRNA and a donor gRNA include the same sequence, such as sharing a nucleic acid sequence capable of hybridizing to both a target sequence to be modified and a CDL target sequence. In other embodiments of the disclosure, a target gRNA and a donor gRNA have different sequences. In the compositions and methods described herein, a gRNA comprises a portion that is complementary to a cognate nucleic acid sequence. Once a gRNA forms an RNP complex with a targetable nuclease (e.g., a first RNA-guided nuclease), the RNP complex can be guided to a cognate nucleic acid sequence by the complementarity between the gRNA and the cognate nucleic acid sequence. In some embodiments, the targetable nuclease is a Cas9 protein. The Cas9 protein “identifies” a cognate nucleic acid sequence by first identifying a 3-base pair protospacer adjacent motif (PAM) located 3′ of the cognate nucleic acid sequence. Once the PAM is identified, a gRNA in the RNP complex hybridizes to the cognate nucleic acid sequence upstream of the PAM. In some embodiments, a gRNA includes a portion of nucleotides that are complementary to a portion in cognate nucleic acid sequence that is approximately 20 nucleotides upstream of the PAM sequence. In general, a Cas9 protein or a variant thereof cleaves about three nucleotides upstream of the PAM sequence. A gRNA can be selected using a software. As a non-limiting example, considerations for selecting a gRNA can include, e.g., the PAM sequence for the RNA-guided nuclease to be used, and strategies for minimizing off-target modifications. Tools, such as NUPACK R and the CRISPR Design Tool, can provide sequences for preparing the gRNA, for assessing target modification efficiency, and/or assessing cleavage at off-target sites.

In some embodiments, a gRNA comprises a portion of at least 17 nucleotides (e.g., 17, 18, 19, 20, 21, 22, 23, 24, or 25 nucleotides) that are complementary to a cognate nucleic acid sequence. In some embodiments, a gRNA can be completely complementary or partially complementary to a cognate nucleic acid sequence. In some embodiments, at least 60% (e.g., 65%, 70%, 75%, 80%, 85%, 90%, 95%, or 97%) of the nucleotides in a cognate nucleic acid sequence can engage in Watson-Crick base pairing with their corresponding nucleotides in a gRNA.

As described in detail further herein, a CDL target sequence and the PAM on one or both termini of the HDR template can be designed with different configurations.

In some embodiments of the disclosure, a target gRNA and a donor gRNA have the same sequence (e.g., a genomic target sequence and a CDL target sequence are the same) and each form a complex with the same species of targetable nuclease (e.g., both have the same sequence and form a RNP complex with the same species of Cas protein). In this case, the gRNA can form a first RNP complex with the RNA-guided nuclease. The first RNP complex can bind to the target nucleic acid via the hybridization between the gRNA and the target nucleic acid. The gRNA can also form a second RNP complex with the RNA-guided nuclease and the donor template. In this second RNP complex, the gRNA can bind to the DNA-binding protein target sequence in the donor template to bring the donor template to the desired intracellular location (e.g., the nucleus) for homologous recombination to occur at the cleaved target nucleic acid. In some embodiments, the gRNA and the DNA-binding protein target sequence only have partial complementarity. In some embodiments of the disclosure, a target gRNA and a donor gRNA have a different sequence (e.g., a genomic target sequence and a CDL target sequence are distinct). Distinct target gRNAs and donor gRNAs can each form a complex with the same species of targetable nuclease (e.g., each form a RNP complex with the same species of Cas protein). Distinct target gRNAs and donor gRNAs can each form a complex with a distinct species of targetable nuclease (e.g., each form a RNP complex with the same species of Cas protein).

### VI. Donor Template

The HDR template, CDL target sequences and corresponding PAM sequences can have several different configurations in a plasmid donor template to enhance homology directed repair between the HDR template and a target nucleic acid. In general, a donor template includes (i) a homology directed repair (HDR) template; (ii) a first co-delivery linearization (CDL) target sequence, wherein the first CDL target sequence is operably linked 5′ of the HDR template; and (iii) a second CDL target sequence, wherein the second CDL target sequence is operably linked 3′ of the HDR template, and wherein each of the first and the second CDL target sequences are capable of cleavage by the targetable nuclease protein or a complex comprising the targetable nuclease protein. In embodiments where the targetable nuclease is a Cas protein (e.g. Cas9), each of the first and the second CDL target sequences are operably linked to a 3-base pair PAM located 3′ of the CDL target sequences. A CDL target sequence and corresponding PAM can be oriented such that the PAM is located between the CDL target sequence and an HDR template. A CDL target sequence and corresponding PAM can be oriented such that the CDL target sequence is located between the PAM and an HDR template. Each CDL target sequence and corresponding PAM in a plasmid donor template can be independently oriented. A PAM corresponding to the first CDL target sequence can be located between the CDL target sequence and an HDR template. The first CDL target sequence can be located between the PAM and an HDR template. A PAM corresponding to the second CDL target sequence can be located between the CDL target sequence and an HDR template. The second CDL target sequence can be located between the PAM and an HDR template. In a non-limiting example, a PAM corresponding to the first CDL target sequence is located between the first CDL target sequence and an HDR template, and a PAM corresponding to the second CDL target sequence is located between the second CDL target sequence and the HDR template.

In some embodiments, the size or length of the plasmid donor template is greater than about 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1 kb, 1.1 kb, 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb, any size of template in between these sizes, or greater than 10 kb. For example, the size of the template can be about 200 bp to about 500 bp, about 200 bp to about 750 bp, about 200 bp to about 1 kb, about 200 bp to about 1.5 kb, about 200 bp to about 2.0 kb, about 200 bp to about 2.5 kb, about 200 bp to about 3.0 kb, about 200 bp to about 3.5 kb, about 200 bp to about 4.0 kb, about 200 bp to about 4.5 kb, about 200 bp to about 5.0 kb. In some cases, the size of the template is large enough and in sufficient quantity to be lethal as naked DNA.

In some embodiments, a donor template encodes a heterologous protein or a fragment thereof. In some embodiments, a donor template includes regulatory sequences, for example, a promoter sequence and/or an enhancer sequence to regulate expression of the heterologous protein or fragment thereof, e.g., after insertion into the genome of a cell. A heterologous protein can include a chimeric antigen receptor (CAR). A heterologous protein can include a T cell receptor (TCR).

In some embodiments, a plasmid donor template includes an exogenous sequence such as an exogenous nucleotide sequence. An exogenous sequence can include an encoded heterologous protein or a fragment thereof. An exogenous sequence can include a gene or portion thereof. An exogenous nucleotide sequence can be a short sequence, e.g., of 3-100 nucleotides in length. An exogenous nucleotide sequence of interest can be a single nucleotide. In addition, an exogenous nucleotide sequence of interest can be a long sequence, e.g., of 500-3000 nucleotides in length. An exogenous nucleotide sequence of interest can be coding or non-coding for a polypeptide sequence. In addition, an exogenous nucleotide sequence of interest can be inserted in a cell such that it forms a chimeric gene upon insertion. For example, an exogenous receptor portion can be inserted in frame in an endogenous receptor coding sequence to produce a chimeric receptor coding sequence that, post-editing, includes the exogenous receptor portion operably linked to an endogenous intracellular portion (e.g., for signal transduction).

In some examples, a gene or portion thereof can be a protein-coding nucleotide sequence (i.e., a nucleotide sequence encoding a polypeptide sequence). In general, any protein coding nucleotide can be used. In some examples, a protein coding nucleotide sequence encodes a protein useful in autologous cell therapies (e.g., autologous T cell therapies). In some examples, a protein coding nucleotide sequence can include, but is not limited to, a factor that modulates the immune system, a cytokine, a factor that modulates T cell function, a factor that promotes T-cell survival, a factor that promotes T-cell function, or an immune checkpoint inhibitor. A protein coding nucleotide sequence, particularly a secreted protein or membrane-bound proteins, can include a nucleotide sequence encoding a signal peptide. The signal peptide can be endogenous to the protein encoded by the protein coding nucleotide sequence. The signal peptide can be exogenous to the protein encoded by the protein coding nucleotide sequence.

In some examples, a gene or portion thereof can be a non-protein coding nucleotide sequence. In general, any non-protein coding nucleotide can be used. In some cases, a non-protein coding nucleotide sequence can be a nucleotide sequence useful in autologous cell therapies (e.g., autologous T cell therapies). In some cases, a non-protein coding nucleotide sequence can include, but is not limited to, an shRNA, an siRNA, an miRNA, and an lncRNA.

Although a nucleotide sequence encoding at least a portion of a gene (e.g., an exogenous gene of interest) can, in general, be any size, practical considerations, such as the impact of gene size on overall template size and on subsequent overall editing efficiency, can be taken into account. Thus, in a particular aspect, provided herein are modified cells that are genomically edited, or are capable of being genomically edited, to express an exogenous gene greater than or equal to 100 bases in length at HR efficiency rates greater than those previously described (e.g., a greater percentage of a population having an integrated polynucleotide sequence), particularly when using non-viral delivery methods. The improved HR efficiency rates similarly apply to genes greater than 100 bases in length, such as introducing exogenous sequences greater than or equal to 200 bases in length, greater than or equal to 400 bases in length, greater than or equal to 500 bases in length, greater than or equal to 600 bases in length, greater than or equal to 750 bases in length, greater than or equal to 1000 bases in length greater than or equal to 1500 bases in length, greater than or equal to 2000 bases in length, greater than or equal to 3000 bases in length, or greater than or equal to 4000 bases in length. The at least a portion of a gene can be greater than or equal to 800 bases in length. The at least a portion of a gene can be greater than or equal to 1600 bases in length.

Exogenous sequences can be between 100-200 bases in length, between 100-300 bases in length, between 100-400 bases in length, between 100-500 bases in length, between 100-600 bases in length, between 100-700 bases in length, between 100-800 bases in length, between 100-900 bases in length, or between 100-1000 bases in length. Exogenous sequences can be between 100-2000 bases in length, between 100-3000 bases in length, between 100-4000 bases in length, between 100-5000 bases in length, between 100-6000 bases in length, between 100-7000 bases in length, between 100-8000 bases in length, between 100-9000 bases in length, or between 100-10,000 bases in length. Exogenous sequences can be between 1000-2000 bases in length, between 1000-3000 bases in length, between 1000-4000 bases in length, between 1000-5000 bases in length, between 1000-6000 bases in length, between 1000-7000 bases in length, between 1000-8000 bases in length, between 1000-9000 bases in length, or between 1000-10,000 bases in length.

Exogenous sequences can be greater than or equal to 10 bases in length, greater than or equal to 20 bases in length, greater than or equal to 30 bases in length, greater than or equal to 40 bases in length, greater than or equal to 50 bases in length, greater than or equal to 60 bases in length, greater than or equal to 70 bases in length, greater than or equal to 80 bases in length greater than or equal to 90 bases in length, or greater than or equal to 95 bases in length. Exogenous sequences can be between 1-100 bases in length, between 1-90 bases in length, between 1-80 bases in length, between 1-70 bases in length, between 1-60 bases in length, between 1-50 bases in length, between 1-40 bases in length, or between 1-30 bases in length. Exogenous sequences can be between 1-20 bases in length, between 2-20 bases in length, between 3-20 bases in length, between 5-20 bases in length, between 10-20 bases in length, or between 15-20 bases in length. Exogenous sequences can be between 1-10 bases in length, between 2-10 bases in length, between 3-10 bases in length, between 5-10 bases in length, between 1-5 bases in length, or between 1-15 bases in length. Exogenous sequences can be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 115, 120, 125, 150, 175, 200, 225, or 250 bases in length. Exogenous sequences can be 1, 2, 3, 4, 5, 6, 7, 8, 9, 10, 11, 12, 13, or 14 bases in length. Exogenous sequences can be greater than about 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1 kb, 1.1 kb, 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb or any size of template in between these sizes.

In examples where multiple exogenous sequences are introduced, the multiple exogenous sequences can be different sizes, e.g., a first exogenous sequence can be greater than or equal to 100 bases and a second exogenous sequence can be greater than or equal to 100 bases, or a first exogenous sequence can be greater than or equal to 100 bases and a second exogenous sequence can be less than 100 bases (e.g., between 1-100 bases in length).

In general, a plasmid donor template is a circular DNA plasmid. In some cases, a plasmid donor template is a double-stranded plasmid. In some cases, a plasmid donor template is a single-stranded plasmid. In some cases, a plasmid donor template is a mini-circle. In some cases, a plasmid donor template is a nanoplasmid.

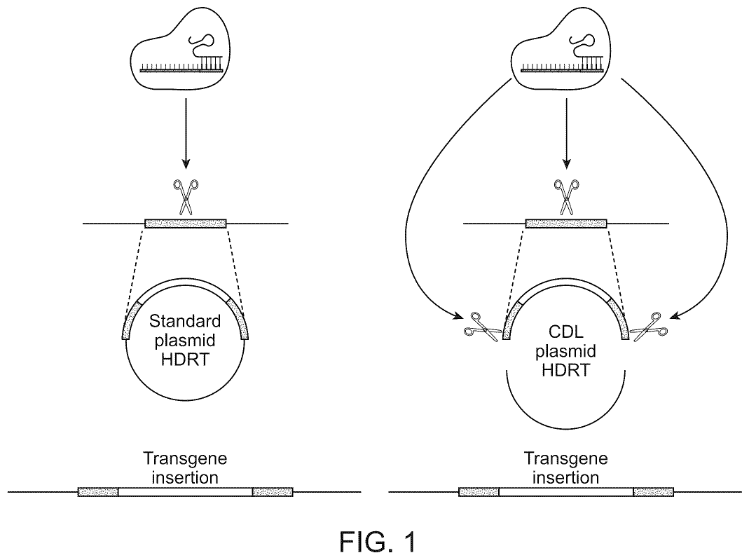

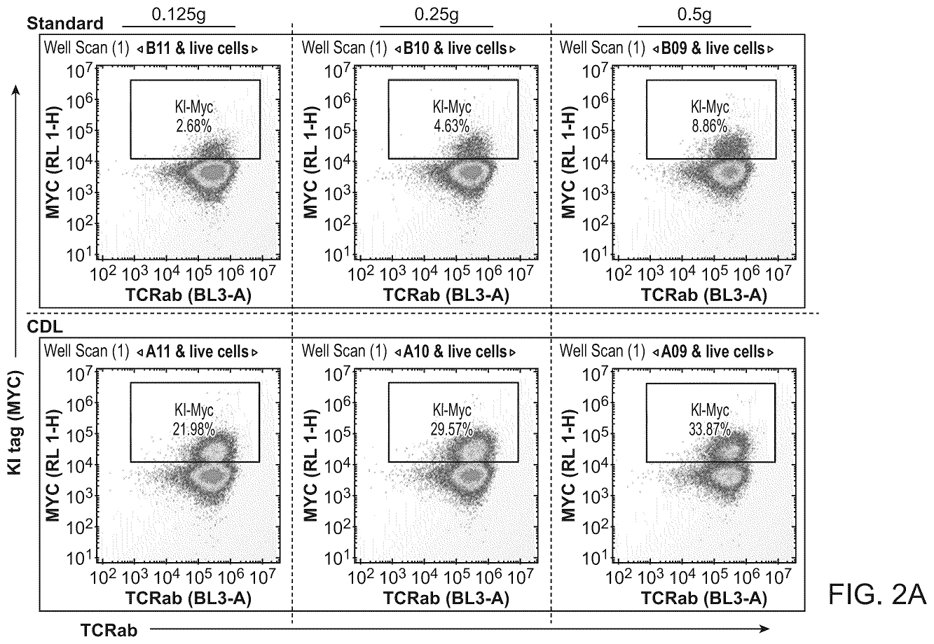

CDL target sequences and/or HDR template components (homology arms, gene of interest, etc.) can be introduced into dsDNA templates of any format, including linear dsDNA sequences produced by PCR, restriction enzyme digestions, or any other linearization method, as well as circular dsDNA sequences such as plasmids. In the case of a plasmid, CDL target sequences can be cloned into the plasmid outside of the homology arms and DNA insert regions, including but not limited to adjacent to the edge(s) of the homology arm(s). Similar to linear dsDNA templates, the DNA binding protein complex (e.g., RNP made from Cas9 and gRNA) can be incubated briefly with plasmid DNA template to allow for binding of the DNA plasmid by the RNP prior to introduction into the cell (e.g., via electroporation). See, e.g., FIGS. 1, 2, and 10B of International Patent Publication No. WO2018232356 and paragraph of International Patent Publication No. WO2019084552.

A plasmid donor template can further contain one or more additional spacer sequences between a CDL target sequence and an HDR template. In some embodiments, a spacer sequence can have at least 2 nucleotides, e.g., between 2 and 24 nucleotides (e.g., between 2 and 22, between 2 and 20, between 2 and 18, between 2 and 16, between 2 and 14, between 2 and 12, between 2 and 10, between 2 and 8, between 2 and 6, between 2 and 4, between 4 and 24, between 6 and 24, between 8 and 24, between 10 and 24, between 12 and 24, between 14 and 24, between 16 and 24, between 18 and 24, between 20 and 24, or between 22 and 24 nucleotides; 2, 3, 4, 5, 6, 7, 8, 9, 10, 11, 12, 13, 14, 15, 16, 17, 18, 19, 20, 21, 22, 23, or 24 nucleotides).

### VII. Targetable Nuclease

As described above, in some embodiments of the compositions and methods described herein, a targetable nuclease is an RNA-guided nuclease (e.g., a Cas protein). A targetable nuclease can recognize a sequence of a cognate nucleic acid sequence (e.g., a target gene within a genome and/or a CDL target sequence), bind to cognate nucleic acid sequence, and modify the cognate nucleic acid sequence. In other embodiments, the targetable nuclease can be a fusion protein that includes a protein that can bind to a cognate nucleic acid sequence and a protein that can modify a cognate nucleic acid sequence (e.g., a nuclease, a transcription activator or repressor).

In some embodiments, a targetable nuclease has nuclease activity. For example, the targetable nuclease can modify a cognate nucleic acid sequence by cleaving the cognate nucleic acid sequence. The cleaved cognate nucleic acid sequence can then undergo homologous recombination (e.g., via HMEJ) with a nearby a homology directed repair (HDR) template (e.g., an HDR template provided a plasmid donor template). For example, a Cas nuclease can direct cleavage of one or both strands at a location in a cognate nucleic acid sequence. Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, Cas9, and Cfp1. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470,

Cas nucleases, e.g., Cas9 nucleases, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp, Succinogenes. Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp, Jejuni. Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

Cas9 protein refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, a Cas9 protein can be a fusion protein, e.g., the two catalytic domains are derived from different bacteria species.

In some embodiments, a Cas protein can be a Cas protein variant. For example, useful variants of the Cas9 nuclease can include a single inactive catalytic domain, such as a RuvC⁻ or HNH⁻ enzyme or a nickase. A Cas9 nickase has only one active functional domain and can cut only one strand of a cognate nucleic acid sequence, thereby creating a single strand break or nick. In some embodiments, a Cas9 nuclease can be a mutant Cas9 nuclease having one or more amino acid mutations. For example, a mutant Cas9 having at least a D10A mutation is a Cas9 nickase. In other embodiments, a mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, a Cas protein variant lacks cleavage (e.g., full cleavage or nickase) activity. A Cas protein variant may contain one or more point mutations that eliminates the protein's nickase activity. In some embodiments, Cas protein variants can be fused to other proteins and serve as targeting domains to direct the other proteins to the target nucleic acid. For example, Cas protein variants without cleavage activity may be fused to transcriptional activation or repression domains to control gene expression (Ma et al., Protein and Cell, 2(11):879-888, 2011; Maeder et al., Nature Methods, 10:977-979, 2013; and Konermann et al., Nature, 517:583-588, 2014). A Cas protein variant that lacks cleavage activity may be used to target genomic regions, resulting in RNA-directed transcriptional control. In some embodiments, a Cas protein variant without any cleavage activity may be used to target an exogenous protein to the target nucleic acid. An exogenous protein may be fused to the Cas protein variant. An exogenous protein may be an effector protein domain. An exogenous protein may be a transcription activator or repressor. Other examples of exogenous proteins include, but are not limited to, VP64-p65-Rta (VPR), VP64, P65, Krab, Ten-eleven translocation methylcytosine dioxygenase (TET), and DNA methyltransferase (DNMT). Specific Cas protein variants that lack cleavage (e.g., nickase) activity are also described below.

In some embodiments, a Cas nuclease can be a high-fidelity or enhanced specificity Cas9) polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9(1.0)), and SpCas9 (K848A/K1003A/R1060A) (also referred to as eSpCas9(1.1)) variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HFI contains all four mutations).

In some embodiments, a targetable nuclease can also be can be a fusion protein that contains a protein that can bind to a cognate nucleic acid sequence and a protein that can cleave the cognate nucleic acid sequence. For example, a protein that can recognize and bind to a cognate nucleic acid sequence can be a Cas protein variant without any cleavage activity. A Cas protein variant without any cleavage activity can be a Cas9 polypeptide that contains two silencing mutations of the RuvC1 and HNH nuclease domains (D10A and H840A), also referred to as dCas9 (Jinek et al., Science, 2012, 337:816-821; Qi et al., Cell, 152(5); 1173-1183). In one embodiment, the dCas9 polypeptide from Streptococcus pyogenes comprises at least one mutation at position D10, G12, G17, E762, H840, N854, N863, H982, H983, A984. D986, A987 or any combination thereof. Descriptions of such dCas9 polypeptides and variants thereof are provided in, for example, International Patent Publication No. WO 2013/176772. The dCas9 enzyme can contain a mutation at D10, E762, H983, or D986, as well as a mutation at H840 or N863. In some instances, the dCas9 enzyme can contain a D10A or DION mutation. Also, the dCas9 enzyme can contain a H840A, H840Y, or H840N. In some embodiments, the dCas9 enzyme can contain D10A and H840A; D10A and H840Y; D10A and H840N; DION and H840A; DION and H840Y; or DION and H840N substitutions. The substitutions can be conservative or non-conservative substitutions to render the Cas9 polypeptide catalytically inactive while still able to bind to a cognate nucleic acid sequence.

In other embodiments, a protein that can recognize and bind to cognate nucleic acid sequence can be a transcription activator-like (TAL) effector DNA-binding protein or a zinc finger DNA-binding protein. The TAL effector DNA-binding protein has a central domain of DNA-binding tandem repeats usually containing 33-35 amino acids in length and two hypervariable amino acid residues at positions 12 and 13 that can recognize one or more specific DNA base pairs. The zinc finger DNA-binding protein has a DNA-binding motif that is often characterized by the absence or presence one or more zinc ions in order to coordinate and stabilize the motif fold. The zinc finger DNA-binding protein contains multiple finger-like protrusions that make tandem contacts with their target molecule. Some zinc finger DNA-binding proteins also form salt bridges to stabilize the finger-like folds. They were first identified as a DNA-binding motif in transcription factor TFIIIA from Xenopus laevis (African clawed frog), however they are now recognized to bind DNA, RNA, protein, and/or lipid substrates.

In some embodiments, a targetable nuclease in the compositions and methods described herein can be a fusion protein containing a TAL effector DNA-binding protein and a protein that can cleave a cognate nucleic acid sequence (also referred to as “Transcription activator-like effector nucleases (TALEN)”). In other embodiments, a targetable nuclease in the compositions and methods described herein can be a fusion protein containing a zinc finger DNA-binding protein and a protein that can cleave cognate nucleic acid sequence. For example, a protein that can cleave cognate nucleic acid sequence can be a wild-type or mutated FokI endonuclease or the catalytic domain of FokI. Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4); 291-303; Gaj et al., Nat Methods, 2012, 9(8); 805-7; Beurdeley et al., Nat Commun, 2013, 4:1762; and Joung and Sander, Nat Rev Mol Cell Biol, 2013, 14(1); 49-55. Examples of a zinc finger DNA-binding protein fused to a protein that can cleave the target nucleic acid are described in the art and include, but are not limited to, those described in Urnov et al., Nature Reviews Genetics, 2010, 11:636-646; Gaj et al., Nat Methods, 2012, 9(8); 805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

In some embodiments, a targetable nuclease does not have nuclease activity. For example, the targetable nuclease (e.g., a targetable nuclease without any nuclease activity) can regulate the expression of a cognate nucleic acid sequence. In some embodiments, the targetable nuclease can be a fusion protein that includes a protein that can bind to cognate nucleic acid sequence, such as a Cas protein variant without any cleavage activity (e.g., a dCas9), a TAL effector DNA-binding protein, and a zinc finger DNA-binding protein as described above, and a protein that can modify the cognate nucleic acid sequence, such as a transcription activator or repressor.

A targetable nuclease can also be fused with a localization peptide or protein. For example, a targetable nuclease can be fused with one or more nuclear localization signal (NLS) sequences, which can direct a targetable nuclease, and a RNP complexes it forms, to the nucleus to modify a cognate nucleic acid sequence. Examples of NLS sequences are known in the art, e.g., as described in Lange et al., J Biol Chem. 282(8):5101-5, 2007, and also include, but are not limited to, AVKRPAATKKAGQAKKKKLD (SEQ ID NO; 3), MSRRRKANPTKLSENAKKLAKEVEN (SEQ ID NO; 4), PAAKRVKLD (SEQ ID NO; 5), KLKIKRPVK (SEQ ID NO; 6), and PKKKRKV (SEQ ID NO; 7). Examples of other peptide or proteins that can be used to a targetable nuclease, such as cell-penetrating peptides and cell-targeting peptides are available in the art and described, e.g., Vivès et al., Biochim Biophys Acta. 1786(2); 126-38, 2008.

A targetable nuclease that modifies a target nucleic acid (e.g., a genomic target sequence, such as in a T cell genome) can be the same as a targetable nuclease capable of cleaving a CDL target sequence. In an illustrative non-limiting example, a targetable nuclease that modifies a target nucleic acid and a targetable nuclease capable of cleaving a CDL target sequence are both a Cas9 protein. A targetable nuclease that modifies a target nucleic acid can be distinct from a targetable nuclease capable of cleaving a CDL target sequence. For example, a targetable nuclease that modifies a target nucleic acid can be a Cas9 protein and a targetable nuclease capable of cleaving a CDL target sequence can be a TALEN, ZFN, or non-Cas9 protein. In another example, a targetable nuclease that modifies a target nucleic acid can be a TALEN, ZFN, or non-Cas) protein and a targetable nuclease capable of cleaving a CDL target sequence can be a Cas9 protein.

A targetable nuclease capable of cleaving a first CDL target sequence can be the same as a targetable nuclease capable of cleaving a second CDL target sequence. In an illustrative non-limiting example, a targetable nuclease capable of cleaving a first CDL target sequence and a targetable nuclease capable of cleaving a second CDL target sequence are both a Cas9 protein. A targetable nuclease capable of cleaving a first CDL target sequence can be distinct from a targetable nuclease capable of cleaving a second CDL target sequence. For example, a targetable nuclease capable of cleaving a first CDL target sequence can be a Cas9 protein and a targetable nuclease capable of cleaving a second CDL target sequence can be a TALEN, ZFN, or non-Cas9 protein. In another example, a targetable nuclease capable of cleaving a first CDL target sequence can be can be a TALEN, ZFN, or non-Cas9 protein and a targetable nuclease capable of cleaving a second CDL target sequence can be a Cas9 protein.

### VIII, CDL Target Sequence

A co-delivery linearization (CDL) target sequence is a nucleotide sequence that is recognized and bound by a targetable nuclease capable of cleaving the CDL target sequence. In the compositions and methods described herein, CDL target sequences flank an HDR template, such that the HDR template can be linearized or excised from a plasmid donor template. Without wishing to be bound by theory, a linearized/excised HDR template can promote homology mediated end joining (HMEJ), while delivery as a plasmid can reduce cytotoxicity. Thus, a CDL target sequence can help to improve knock-in cell yield, such as through improved homology directed repair efficiency and/or reduced cytotoxicity.

In some embodiments, a CDL target sequence can be directly recognized and bound by a DNA-binding protein, e.g., a TAL effector DNA-binding protein or zinc finger DNA-binding protein. In other embodiments, a CDL target sequence can be indirectly recognized and bound by a DNA-binding protein, e.g., an RNA-guided nuclease, via a donor gRNA. In some embodiments, at least 60% (e.g., 65%, 70%, 75%, 80%, 85%, 90%, 95%, or 97%) of the nucleotides in a CDL target sequence can engage in Watson-Crick base pairing with their corresponding nucleotides in the donor gRNA. In some embodiments, a CDL target sequence can have at least one (e.g., one, two, three, four, five, six, seven, eight, nine, or ten) mismatched nucleotide to its corresponding nucleotide in a donor gRNA when the CDL target sequence and a donor gRNA are hybridized. Examples of mismatched bases include a guanine and uracil, guanine and thymine, and adenine and cytosine pairing. In some embodiments, a CDL target sequence is a portion of a target nucleic acid (e.g., a genomic target of a cell).

In general, CDL target sequences are present at both termini of an HDR template in a plasmid donor template as described above. A CDL target sequence and, when a Cas system, a corresponding PAM in a plasmid donor template can have different configurations as described above. In some embodiments, a CDL target sequence is complementary to an equal length portion of the sequence of a donor gRNA. In some embodiments, a CDL target sequence has at least 17 nucleotides, e.g., between 17 and 20 nucleotides (e.g., between 17 and 19, between 17 and 18, between 18 and 20, between 18 and 19, or 17, 18, 19, or 20 nucleotides). In some embodiments, a CDL target sequence is partially complementarity, i.e., comprises nucleotide mismatches, compared to an equal length portion of the sequence of a donor gRNA. For example, a DNA-binding protein target sequence having 20 nucleotides can have between 1 and 6 nucleotide mismatches (e.g., between 1 and 5, between 1 and 4, between 1 and 3, between 1 and 2 nucleotide mismatches; 1, 2, 3, 4, 5, or 6 nucleotide mismatches) compared to a 20-nucleotide portion of the sequence of a donor gRNA.

### IX. Gene Targeting Nucleic Acids in Cells

The compositions described herein can be used in methods of modifying a target nucleic acid in a cell, e.g., an eukaryotic cells, prokaryotic cell, animal cell, plant cell, fungal cell, and the like. Optionally, the cell is a mammalian cell, for example, a human cell. The cell can be in vitro, ex vivo, or in vivo. The cell can also be a primary cell, a germ cell, a stem cell, or a precursor cell. The precursor cell can be, for example, a pluripotent stem cell, or a hematopoietic stem cell. In some embodiments, the cell is a primary hematopoietic cell, a primary hematopoietic stem cell, or a primary T cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4⁺ T cell. In some embodiments, the T cell is a CD8⁺ T cell. In some embodiments, the T cell is a CD4⁺ CD8⁺ T cell. In some embodiments, the T cell is a CD4⁻ CD8⁻-T cell. In some embodiments the T cell is an αβ T cell, in some embodiments the T cell is a γδ T cell. Populations of any of the cells modified by any of the methods described herein are also provided. In some embodiments, the methods further comprise expanding the population of modified cells.

In a particular aspect, a population of cells (e.g., a population of T cells), is provided. The population of cells can comprise any of the modified cells described herein. The modified cell can be within a heterogeneous population of cells and/or a heterogeneous population of different cell types. The population of cells can be heterogeneous with respect to the percentage of cells that are genomically edited. A population of cells can have greater than 10%, greater than 20%, greater than 30%, greater than 40%, greater than 50%, greater than 60%), greater than 70%, greater than 80%, or greater than 90% of the population comprise an integrated nucleotide sequence. In a certain aspect, a populations of cells comprises an integrated nucleotide sequence, wherein the integrated nucleotide sequence comprises at least a portion of a gene, the integrated nucleotide sequence is integrated at an endogenous genomic target locus, and the integrated nucleotide sequence is orientated such that the at least a portion of the gene is capable of being expressed, wherein the population of cells is substantially free of viral-mediated delivery components, and wherein greater than 10%), greater than 20%, greater than 30%, greater than 40%, greater than 50%, greater than 60%), greater than 70%, greater than 80%, or greater than 90% of the cells in the population comprise the integrated nucleotide sequence.

A population of cells can have greater than 91%, greater than 92%, greater than 93%), greater than 94%, greater than 95%, greater than 96%, or greater than 97%, greater than 98%), greater than 99%, greater than 99.5%, or greater than 99.9% of the population comprise an integrated nucleotide sequence. A population of cells can have greater than 20% of the population comprise an integrated nucleotide sequence. A population of cells can have greater than 30% of the population comprise an integrated nucleotide sequence. A population of cells can have greater than 60% of the population comprise an integrated nucleotide sequence. A population of cells can have greater than 70% of the population comprise an integrated nucleotide sequence.

A cell can include a cell comprising a non-virally inserted sequence such as an exogenous sequence. A cell can be virus-free. A cell can be substantially free of virus. A cell can include at least one nucleic acid sequence (e.g., comprising at least one heterologous gene) non-virally inserted into at least one target region. In certain aspects, a cell does not comprise a viral vector, e.g., for introducing at least one nucleic acid sequence such as a donor template.

A cell can include one or more primary cells that include a non-virally inserted exogenous sequence that is at least 200 base pairs in size. The primary cells can be primary hematopoietic cells or primary hematopoietic stem cells. The primary cells can be primary hematopoietic cells and the primary hematopoietic cells can be immune cells. The immune cells can be T cells. The primary cells can be human cells. In some aspects, the primary cells do not comprise a viral vector. The size of the exogenous sequence can be greater than a length selected from the group consisting of; 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1 kb, 1.1 kb, 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, and 5.0 kb. The size of the exogenous sequence can be greater than 1.5 kb. The size of the exogenous sequence can be about 200 bp to about 500 bp, about 200 bp to about 750 bp, about 200 bp to about 1 kb, about 200 bp to about 1.5 kb, about 200 bp to about 2.0 kb, about 200 bp to about 2.5 kb, about 200 bp to about 3.0 kb, about 200 bp to about 3.5 kb, about 200 bp to about 4.0 kb, about 200 bp to about 4.5 kb, about 200 bp to about 5.0 kb. The size of the exogenous sequence can be greater than about 1 kb. The exogenous sequence can include a regulatory sequence, optionally wherein the regulatory sequence comprises a promoter sequence and/or an enhancer sequence.

The exogenous sequence can encode a heterologous protein or a fragment thereof. The exogenous sequence can encode a chimeric antigen receptor (CAR). The exogenous sequence can encode a T cell receptor (TCR).

A cell can include a primary human T cell comprising a non-virally inserted DNA template having a size of greater than 1 kb,

A cell can include a primary human T cell comprising: at least one nucleic acid sequence comprising at least one heterologous gene non-virally inserted into at least one target region of one or both of: endogenous T cell receptor alpha subunit constant gene (TRAC), and endogenous T cell receptor beta subunit constant gene (TRBC), the at least one heterologous gene comprises at least one of: (1) a variable region of a heterologous T cell receptor alpha (TCR-a) chain gene and (2) a variable region of a heterologous T cell receptor beta (TCR-B) chain gene. In some aspects, the T cell does not comprise a viral vector for introducing the at least one nucleic acid sequence to the T cell. In some aspects, the at least one nucleic acid sequence is at least 1.5 kb in size. In some aspects, the at least one nucleic acid sequence is at least 500 bp in size. In some aspects, the target region is in exon 1, 2, or 3 of TRAC. In some aspects, the target region is in exon 1, 2, or 3 of TRBC. In some aspects, the T cell is a CD8+ T cell or a CD4+ T cell. In some aspects, the at least one heterologous gene comprises at least one of: (1) a) variable region or b) variable region and constant region of the heterologous T cell receptor alpha (TCR-α) chain gene and (2) a) variable region or b) variable region and constant region of the heterologous T cell receptor beta (TCR-β) chain gene. In some aspects, the at least one heterologous gene comprises each of: (1) the a) variable region or b) variable region and constant region of the heterologous T cell receptor alpha (TCR-α) chain gene and (2) the a) variable region or b) variable region and constant region of the heterologous T cell receptor beta (TCR-B) chain gene. In some aspects, the T cell comprises each of (1) the a) variable region or b) variable region and constant region of the heterologous TCR-a chain gene and (2) the a) variable region or b) variable region and constant region of the heterologous TCR-β chain gene. In some aspects, the heterologous genes form an antigen-specific T cell receptor (TCR) upon expression. In some aspects, the heterologous TCR-a chain gene and the heterologous TCR-β chain gene are operably linked by a linker sequence, optionally the linker sequence is a cleavable linker sequence or a multicistronic element. In some aspects, the heterologous TCR-a chain gene and the heterologous TCR-β chain gene are inserted into TRAC. In some aspects, expression of the at least one heterologous gene is driven by an endogenous promoter. In some aspects, expression of one or both of TRAC and TRBC is reduced in the cell relative to a control T cell, wherein the control T cell is a primary human T cell that lacks the non-viral insertion.

A cell can include a primary human T cell comprising: at least one nucleic acid sequence comprising at least one heterologous gene inserted into at least one target region of one or both of: endogenous T cell receptor alpha subunit constant gene (TRAC), and endogenous T cell receptor beta subunit constant gene (TRBC), the at least one heterologous gene comprises at least one of: (1) a variable region of a heterologous T cell receptor alpha (TCR-α) chain gene and (2) a variable region of a heterologous T cell receptor beta (TCR-B) chain gene, and wherein the T cell does not comprise a viral vector for introducing the at least one nucleic acid sequence to the T cell.

A cell can include a primary cell comprising: at least one nucleic acid sequence comprising at least one heterologous gene non-virally inserted into at least one target region of the cell's genome. In some aspects the at least one heterologous gene encodes a CAR or other chimeric receptor. In some aspects the at least one heterologous gene comprises at least one or both of: (1) a variable region of a heterologous T cell receptor alpha (TCR-α) chain gene and (2) a variable region of a heterologous T cell receptor beta (TCR-B) chain gene. In some aspects the primary cell is a T cell. In some aspects, the cell does not comprise a viral vector for introducing the at least one nucleic acid sequence to the cell. In some aspects, the at least one nucleic acid sequence is at least 1.5 kb in size. In some aspects, the at least one nucleic acid sequence is at least 500 bp in size. In some aspects, the target region is in TRAC, e.g., exon 1, 2, or 3 of TRAC. In some aspects, the target region is in TRBC, e.g., exon 1, 2, or 3 of TRBC. In some aspects, the T cell is a CD8+ T cell or a CD4+ T cell. In some aspects, expression of the at least one heterologous gene is driven by an endogenous promoter. In some aspects, expression of one or both of TRAC and TRBC is reduced in the T cell relative to a control T cell, wherein the control T cell is a primary human T cell that lacks the non-viral insertion.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors. In some embodiments, the chimeric antigen receptor includes an extracellular portion containing an antibody or fragment described herein. In some aspects, the chimeric antigen receptor includes an extracellular portion containing an antibody or fragment described herein and an intracellular signaling domain. In some embodiments, an antibody or fragment includes an scFv or a single-domain VH antibody and the intracellular domain contains an ITAM. In some aspects, the intracellular signaling domain includes a signaling domain of a ζchain of a CD3 (CD3ζ chain). In some embodiments, the chimeric antigen receptor includes a transmembrane domain linking the extracellular domain and the intracellular signaling domain. In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB. In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer. In some embodiments, the transmembrane domain of the receptor, e.g., the CAR, is a transmembrane domain of human CD28 or variant thereof, e.g., a 27-amino acid transmembrane domain of a human CD28 (Accession No.: P10747.1). In some embodiments, the chimeric antigen receptor contains an intracellular domain of a T cell costimulatory molecule. In some aspects, the T cell costimulatory molecule is CD28 or 41BB. In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof. In some embodiments, the intracellular signaling domain comprises a human CD3 zeta stimulatory signaling domain or functional variant thereof, such as an 112 AA cytoplasmic domain of isoform 3 of human CD3ζ(Accession No.: P20963.2) or a CD3ζ signaling domain as described in U.S. Pat. No. 7,446,190 or U.S. Pat. No. 8,911,993.

Methods for modifying a target nucleic acid in a cell described herein comprise introducing into the cell a composition described herein, wherein the HDR template is integrated into the target nucleic acid. As demonstrated in the Examples, pre-incubation of a targetable nuclease (e.g., an RNA guided nuclease) and donor gRNA RNP complex with a donor template (comprising a CDL target sequence-modified HDR template) before introducing the composition into the cell enhances knock-in cell yield. In some embodiments, a composition described herein is introduced into the cell via electroporation.

In some cases, the cells are removed from a subject, modified using any of the methods described herein and administered to the subject. In other cases, a composition described herein can be delivered to the subject in vivo. See, for example, U.S. Pat. No. 9,737,604 and Zhang et al. “Lipid nanoparticle-mediated efficient delivery of CRISPR/Cas9) for tumor therapy,” NPG Asia Materials Volume 9, page e441 (2017).

In particular embodiments, the compositions described herein can be used in methods of modifying a target nucleic acid in a primary cell. The compositions described herein can be used in methods for inducing a stable gene modification of a target nucleic acid in a primary cell. In some embodiments, the method includes introducing into the primary cell a composition comprising a Cas protein (e.g., a Cas) protein), one or more single guide RNAs (sgRNAs), and an anionic polymer. The sgRNA may comprise a first nucleotide sequence that is complementary to the target nucleic acid and a second nucleotide sequence that interacts with the Cas protein (e.g., Cas) protein). In some embodiments, a Cas protein (e.g., a Cas) protein) and an sgRNA may be incubated together to form a RNP complex prior to introducing into the primary cell. A composition comprising a Cas protein (e.g., a Cas) protein), one or more single guide RNAs (sgRNAs), and an plasmid donor template may be electroporated into the primary cell. In some embodiments, the primary cell is selected from the group consisting of an immune cell (e.g., a primary T cell), a blood cell, a progenitor or stem cell thereof, a mesenchymal cell, and a combination thereof. In some instances, the immune cell is selected from the group consisting of a T cell, a B cell, a dendritic cell, a natural killer cell, a macrophage, a neutrophil, an eosinophil, a basophil, a mast cell, a precursor thereof, and a combination thereof. The progenitor or stem cell can be selected from the group consisting of a hematopoietic progenitor cell, a hematopoietic stem cell, and a combination thereof. In some cases, the blood cell is a blood stem cell. In some instances, the mesenchymal cell is selected from the group consisting of a mesenchymal stem cell, a mesenchymal progenitor cell, a mesenchymal precursor cell, a differentiated mesenchymal cell, and a combination thereof. The differentiated mesenchymal cell can be selected from the group consisting of a bone cell, a cartilage cell, a muscle cell, an adipose cell, a stromal cell, a fibroblast, a dermal cell, and a combination thereof. In some embodiments, the primary cell can comprise a population of primary cells. In some cases, the population of primary cells comprises a heterogeneous population of primary cells. In other cases, the population of primary cells comprises a homogeneous population of primary cells.

In some embodiments, the primary cell is isolated from a mammal prior to introducing a composition described herein into the primary cell. For instance, the primary cell can be harvested from a human subject. In some instances, the primary cell or a progeny thereof is returned to the mammal after introducing the composition described herein into the primary cell. In other words, the genetically modified primary cell undergoes autologous transplantation. In other instances, the genetically modified primary cell undergoes allogeneic transplantation. For example, a primary cell that has not undergone stable gene modification is isolated from a donor subject, and then the genetically modified primary cell is transplanted into a recipient subject who is different than the donor subject.

A composition described herein can be introduced into a cell (e.g., a primary cell) using available methods and techniques in the art. Non-limiting examples of suitable methods include electroporation, particle gun technology, and direct microinjection. In some embodiments, the step of introducing the composition described herein into the cell comprises electroporating the composition into the cell.

In some embodiments, the stable gene modification of the target nucleic acid is induced in greater than about 5% of the population of cells (e.g., the population of primary cells), e.g., about 6%, about 7%, about 8%, about 9%, about 10%, about 12%, about 14%, about 16%, about 18%, about 20%, about 22%, about 24%, about 26%, about 28%, or about 30% of the population of cells. In some embodiments, the stable gene modification of the target nucleic acid is induced in greater than about 50% of the population of cells (e.g., the population of primary cells), e.g., about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the population of cells. In other embodiments, the stable gene modification of the target nucleic acid is induced in greater than about 70% of the population of cells, e.g., about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the population of cells. In yet other embodiments, the stable gene modification of the target nucleic acid is induced in greater than about 90% of the population of cells, e.g., about 90%, about 91%, about 92%, about 93%, about 94%, about 95%, about 96%, about 97%, about 98%, about 99%, or about 100% of the population of cells.

In other embodiments, the stable gene modification of the target nucleic acid comprises the replacement of a genetic mutation in the target nucleic acid (e.g., to correct a point mutation or a single nucleotide polymorphism (SNP) in the target nucleic acid that is associated with a disease) or the insertion of an open reading frame (ORF) comprising a normal copy of the target nucleic acid (e.g., to knock in a wild-type cDNA of the target nucleic acid that is associated with a disease).

In some embodiments, any of the methods described herein can also include purifying the cell (e.g., a primary cell) having the stable gene modification of the target nucleic acid. In some cases, the composition isolated by the purifying step includes at least about 80% cells having the stable gene modification of the target nucleic acid, e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more cells having the stable gene modification of the target nucleic acid.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

The following examples are provided by way of illustration only and not by way of limitation. Those of skill in the art will readily recognize a variety of non-critical parameters that could be changed or modified to yield essentially the same or similar results.

### Example 1—Experimental Methods

### T Cell Culturing

T-cells were enriched from peripheral blood mononuclear cells (PBMCs) prepared using Lymphoprep (STEMCELL Technologies) from normal donor Leukopaks (STEMCELL Technologies) using the Easy Sep Human T-Cell Isolation Kit (STEMCELL Technologies). T-cells were subsequently activated with CD3/CD28 Dynabeads at 1:1 bead to cell ratio (ThermoFisher, 40203D) in TexMACS medium (Miltenyi 130-197-196) supplemented with 3% human AB serum (Gemini Bio) and 12.5 ng/ml human IL-7 and IL-15 (Miltenyi premium grade) and grown at 37° C., 5% CO2 for 48 hours before electroporation.

### RNP Preparation and T Cell Electroporation

CRISPR RNP were prepared by combining 120 μM sgRNA (Synthego) targeting DNA sequence AAGTCTCTCAGCTGGTACA (SEQ ID NO:1), 62.5 μM sNLS-SpCas9-sNLS protein (Aldevron) and P3 buffer (Lonza) at a ratio of 5:1:3:6. A range of masses (final concentration ranged between 0-100 mg/l) of plasmid DNA (plasmids having HDR templates [an ˜5.7 kb insert encoding a transgene having a Myc epitope-tag and 450 bp flanking homology arms] with or without CDL-sequences flanking HDR homology arms) was mixed with 3.5 μl of RNP. T-cells were counted, centrifuged at 90×G for 10 minutes and resuspended at 10\hat{ }6 cells/14.5 μl of P3 with supplement added (Lonza). 14.5 μl of T-cell suspension was added to the DNA/RNP mixture, transferred to a well on a Lonza 384-well Nucleocuvette Plate, and pulsed in a Lonza HT Nucleofector System with code EH-115. Cells were allowed to rest for 15 minutes at room temperature before transfer to 96-well plates (Sarstedt) in TexMACS medium supplemented with 12.5 ng/ml human IL-7 and IL-15 (Miltenyi premium grade).

### Flow Cytometry Analysis

Transgene expression (gene of interest with a Myc epitope-tag) was detected by staining with anti-Myc antibody (Cell Signaling Technology clone 9B11) and analysis on an Attune N×T Flow Cytometer. Other antibodies used were TCRalpha/beta antibody (BioLegend clone IP26), CD4 antibody (BioLegend clone RPA-T4), CD8 antibody (BioLegend clone SK1).

### Sequences

CRISPR protospacer (e.g., CDL target sequence):

(SEQ ID NO: 2)
GAGCCATGCTTGGCTTACGA

Full CRISPR site, protospacer and exemplary PAM:

(SEQ ID NO: 8)
GAGCCATGCTTGGCTTACGAGGG

Standard plasmid donor backbone sequence used (X indicates where our transgene insertion of length 5696 bp was; Standard uppercase indicates homology arms):

(SEQ ID NO: 9)
CGACCAACCCATCAAACTCCCCGCCCCCAGCACTTTTATTTCTCCTCTTTAGGAA
GTACACTTCAGTATCTTTGGCACAGTGCATGAGCACGACTAAAGTAAAACATCGC
AGAAAACATAGCTTTAGTCTACCCTTCGTGTCCTAAAAGGAAAACCAGTAGCTTC
CCAGGCCACCGGAAGGGCAACACATGTCCTCTGCAGTTTCTGCACACGGGAAGG
TAAAGACAGAGAGAGGACCTACTCCTCAACACAGAAACATTTCAAAATCTTTCC
TCGCCTGCAACCCAAGCTGAAGTCATTCTCCCCAGAAATAACAAAAGTTGGAAG
AGAAGCCGGAGACAGGATAGGTGCAGGAAGCCCACACTTTGAGGGCAGCACTC
AGACACCCTCTCCTGTGTGCAGGACGTGCCGAATGTTCAGGTGCAATGAGAATG
AGCCATGCTTGGCTTA-X-
(SEQ ID NO: 10)
CGAGGGCAATCTGGCCCATCAAGTGGCCTTCGCCTCTGGGAGTAACAAAAATGC
ACTTCAAAATAGCTTCTGTAATCAAGCTGCATGGGTGGAGTACTCCCCAGCTGAC
TCCAGGAAGTTCTCTATCCAAAGCTATTCATTAGGCCAGAGCTGTGCAAATAATT
AGTCACCCACTTGCTCCATAACCCTCCATGACAGCCCAGGCATTGAGTCCAGGTG
GGACCATCAAGCCATGCTCTGGTGGCTCATGCATTATCATAGAAATGGGAGGCTT
TATTTATTTTACTAAAAAGAACAAAAACAACAGACTGCTGTCCTTTAGACAATAG
GATCACGTCATCTGAGCCCTCTGTGCCCCAGGTGACAAGCCCAGCCCCAAGTTCT
CTTTCCTCAGCCTCCCCACACATGTTCTGGAGGAGATGGGCCCAGCAGGCTGCTC
TGAGGCCTGGCatcccaatggcgcgccgagcttggcgtaatcatggtcatagctgtttcctgtgtgaaattgttatccgctcac
aattccacacaacatacgagccggaagcataaagtgtaaagcctggggtgcctaatgagtgagctaactcacattaattgcgttgcgct
cactgcccgctttccagtcgggaaacctgtcgtgccagctgcattaatgaatcggccaacgcgcggggagaggcggtttgcgtattgg
gcgctgttccgcttcctcgctcactgactcgctgcgctcggtcgttcggctgcggcgagcggtatcagctcactcaaaggcggtaatac
ggttatccacagaatcaggggataacgcaggaaagaacatgtgagcaaaaggccagcaaaaggccaggaaccgtaaaaaggccg
cgttgctggcgtttttccataggctccgcccccctgacgagcatcacaaaaatcgacgctcaagtcagaggtggcgaaacccgacag
gactataaagataccaggcgtttccccctggaagctccctcgtgcgctctcctgttccgaccctgccgcttaccggatacctgtccgcct
ttctcccttcgggaagcgtggcgctttctcatagctcacgctgtaggtatctcagttcggtgtaggtcgttcgctccaagctgggctgtgtg
cacgaaccccccgttcagcccgaccgctgcgccttatccggtaactatcgtcttgagtccaacccggtaagacacgacttatcgccact
ggcagcagccactggtaacaggattagcagagcgaggtatgtaggcggtgctacagagttcttgaagtggtggcctaactacggcta
cactagaagaacagtatttggtatctgcgctctgctgaagccagttaccttcggaaaaagagttggtagctcttgatccggcaaacaaac
caccgctggtagcggtggtttttttgtttgcaagcagcagattacgcgcagaaaaaaaggatctcaagaagatcctttgatcttttctacg
gggtctgacgctcagtggaacgaaaactcacgttaagggattttggtcatgagattatcaaaaaggatcttcacctagatccttttaaatta
aaaatgaagttttaaatcaatctaaagtatatatgagtaaacttggtctgacagttaccaatgcttaatcagtgaggcacctatctcagcgat
ctgtctatttcgttcatccatagttgcctgactccccgtcgtgtagataactacgatacgggagggcttaccatctggccccagtgctgca
atgataccgcgagaaccacgctcaccggctccagatttatcagcaataaaccagccagccggaagggccgagcgcagaagtggtc
ctgcaactttatccgcctccatccagtctattaattgttgccgggaagctagagtaagtagttcgccagttaatagtttgcgcaacgttgttg
ccattgctacaggcatcgtggtgtcacgctcgtcgtttggtatggcttcattcagctccggttcccaacgatcaaggcgagttacatgatc
ccccatgttgtgcaaaaaagcggttagctccttcggtcctccgatcgttgtcagaagtaagttggccgcagtgttatcactcatggttatg
gcagcactgcataattctcttactgtcatgccatccgtaagatgcttttctgtgactggtgagtactcaaccaagtcattctgagaatagtgt
atgcggcgaccgagttgctcttgcccggcgtcaatacgggataataccgcgccacatagcagaactttaaaagtgctcatcattggaaa
acgttcttcggggcgaaaactctcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaataagggcgacacggaaatgt
tgaatactcatactcttcctttttcaatattattgaagcatttatcagggttattgtctcatgagcggatacatatttgaatgtatttagaaaa
ataaacaaataggggttccgcgcacatttccccgaaaagtgccacctgacgtctaagaaaccattattatcatgacattaacctataaaaata
ggcgtatcacgaggcccttttgtctcgcgcgtttcggtgatgacggtgaaaacctctgacacatgcagctcccggagactgtcacagct
tgtctgtaagcggatgccgggagcagacaagcccgtcagggcgcgtcagcgggtgttggcgggtgtcggggctggcttaactatgc
ggcatcagagcagattgtactgagagtgcaccatatgcggtgtgaaataccgcacagatgcgtaaggagaaaataccgcatcaggcg
ccattcgccattcaggctgcgcaactgttgggaagggcgatcggtgcgggcctcttcgctattacgccagctggcgaaagggggatg
tgctgcaaggcgattaagttgggtaacgccagggttttcccagtcacgacgttgtaaaacgacggccagtgaattgacgcgtattggga
t

CDL plasmid donor backbone sequence used (X indicates where our transgene insertion of length 5696 bp was: Bold indicates protospacer (CDL target sequence): Lowercase italic indicates PAM: Standard uppercase indicates homology arms):

(SEQ ID NO: 11)
GAGCCATGCTTGGCTTACGAgggCGACCAACCCATCAAACTCCCCGCCCCCAGC
ACTTTTATTTCTCCTCTTTAGGAAGTACACTTCAGTATCTTTGGCACAGTGCATGA
GCACGACTAAAGTAAAACATCGCAGAAAACATAGCTTTAGTCTACCCTTCGTGTC
CTAAAAGGAAAACCAGTAGCTTCCCAGGCCACCGGAAGGGCAACACATGTCCTC
TGCAGTTTCTGCACACGGGAAGGTAAAGACAGAGAGAGGACCTACTCCTCAACA
CAGAAACATTTCAAAATCTTTCCTCGCCTGCAACCCAAGCTGAAGTCATTCTCCC
CAGAAATAACAAAAGTTGGAAGAGAAGCCGGAGACAGGATAGGTGCAGGAAGC
CCACACTTTGAGGGCAGCACTCAGACACCCTCTCCTGTGTGCAGGACGTGCCGAA
TGTTCAGGTGCAATGAGAATGAGCCATGCTTGGCTTA-X-
(SEQ ID NO: 12)
CGAGGGCAATCTGGCCCATCAAGTGGCCTTCGCCTCTGGGAGTAACAAAAATGC
ACTTCAAAATAGCTTCTGTAATCAAGCTGCATGGGTGGAGTACTCCCCAGCTGAC
TCCAGGAAGTTCTCTATCCAAAGCTATTCATTAGGCCAGAGCTGTGCAAATAATT
AGTCACCCACTTGCTCCATAACCCTCCATGACAGCCCAGGCATTGAGTCCAGGTG
GGACCATCAAGCCATGCTCTGGTGGCTCATGCATTATCATAGAAATGGGAGGCTT
TATTTATTTTACTAAAAAGAACAAAAACAACAGACTGCTGTCCTTTAGACAATAG
GATCACGTCATCTGAGCCCTCTGTGCCCCAGGTGACAAGCCCAGCCCCAAGTTCT
CTTTCCTCAGCCTCCCCACACATGTTCTGGAGGAGATGGGCCCAGCAGGCTGCTC
TGAGGCCTGGCcccTCGTAAGCCAAGCATGGCTCatcccaatggcgcgccgagcttggcgtaatcat
ggtcatagctgtttcctgtgtgaaattgttatccgctcacaattccacacaacatacgagccggaagcataaagtgtaaagcctggggtg
cctaatgagtgagctaactcacattaattgcgttgcgctcactgcccgctttccagtcgggaaacctgtcgtgccagctgcattaatgaat
cggccaacgcgcggggagaggcggtttgcgtattgggcgctgttccgcttcctcgctcactgactcgctgcgctcggtcgttcggctg
cggcgagcggtatcagctcactcaaaggcggtaatacggttatccacagaatcaggggataacgcaggaaagaacatgtgagcaaa
aggccagcaaaaggccaggaaccgtaaaaaggccgcgttgctggcgtttttccataggctccgcccccctgacgagcatcacaaaa
atcgacgctcaagtcagaggtggcgaaacccgacaggactataaagataccaggcgtttccccctggaagctccctcgtgcgctctc
ctgttccgaccctgccgcttaccggatacctgtccgcctttctcccttcgggaagcgtggcgctttctcatagctcacgctgtaggtatctc
agttcggtgtaggtcgttcgctccaagctgggctgtgtgcacgaaccccccgttcagcccgaccgctgcgccttatccggtaactatcg
tcttgagtccaacccggtaagacacgacttatcgccactggcagcagccactggtaacaggattagcagagcgaggtatgtaggcgg
tgctacagagttcttgaagtggtggcctaactacggctacactagaagaacagtatttggtatctgcgctctgctgaagccagttaccttc
ggaaaaagagttggtagctcttgatccggcaaacaaaccaccgctggtagcggtggtttttttgtttgcaagcagcagattacgcgcag
aaaaaaaggatctcaagaagatcctttgatcttttctacggggtctgacgctcagtggaacgaaaactcacgttaagggattttggtcatg
agattatcaaaaaggatcttcacctagatccttttaaattaaaaatgaagttttaaatcaatctaaagtatatatgagtaaacttggtctgaca
gttaccaatgcttaatcagtgaggcacctatctcagcgatctgtctatttcgttcatccatagttgcctgactccccgtcgtgtagataacta
cgatacgggagggcttaccatctggccccagtgctgcaatgataccgcgagaaccacgctcaccggctccagatttatcagcaataaa
ccagccagccggaagggccgagcgcagaagtggtcctgcaactttatccgcctccatccagtctattaattgttgccgggaagctaga
gtaagtagttcgccagttaatagtttgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctccttcggtcctccgatcgttgtc
agaagtaagttggccgcagtgttatcactcatggttatggcagcactgcataattctcttactgtcatgccatccgtaagatgcttttctgtg
actggtgagtactcaaccaagtcattctgagaatagtgtatgcggcgaccgagttgctcttgcccggcgtcaatacgggataataccgc
gccacatagcagaactttaaaagtgctcatcattggaaaacgttcttcggggcgaaaactctcaaggatcttaccgctgttgagatccag
ttcgatgtaacccactcgtgcacccaactgatcttcagcatcttttactttcaccagcgtttctgggtgagcaaaaacaggaaggcaaaat
gccgcaaaaaagggaataagggcgacacggaaatgttgaatactcatactcttcctttttcaatattattgaagcatttatcagggttattgt
ctcatgagcggatacatatttgaatgtatttagaaaaataaacaaataggggttccgcgcacatttccccgaaaagtgccacctgacgtct
aagaaaccattattatcatgacattaacctataaaaataggcgtatcacgaggcccttttgtctcgcgcgtttcggtgatgacggtgaaaa
cctctgacacatgcagctcccggagactgtcacagcttgtctgtaagcggatgccgggagcagacaagcccgtcagggcgcgtcag
cgggtgttggcgggtgtcggggctggcttaactatgcggcatcagagcagattgtactgagagtgcaccatatgcggtgtgaaatacc
gcacagatgcgtaaggagaaaataccgcatcaggcgccattcgccattcaggctgcgcaactgttgggaagggcgatcggtgcggg
cctcttcgctattacgccagctggcgaaagggggatgtgctgcaaggcgattaagttgggtaacgccagggttttcccagtcacgacgt
tgtaaaacgacggccagtgaattgacgcgtattgggat

### Example 2—Co-delivery Linearization (CDL) Designs for CRISPR-mediated Gene Editing of T Cells

Genetic engineering of T cells using RNP electroporation was explored comparing a plasmid donor templates with (1) a homology-directed repair template cassette (HDRT) featuring a standard HDRT design of a transgene simply flanked by homology arms (FIG. 1—left panel) or (2) a co-delivery linearization (CDL) design where the HDRT was additionally flanked with CDL target sequences (CRISPR target sites with a match to the protospacer of the CRISPR gRNA used to target the T cell genome) and protospacer-adjacent motif (PAM) (FIG. 1—right panel).

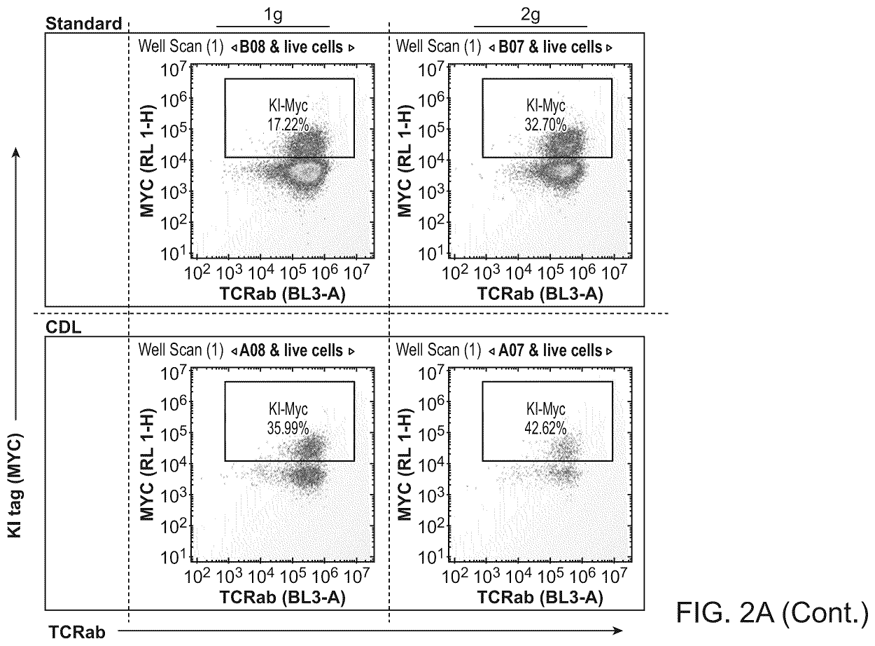

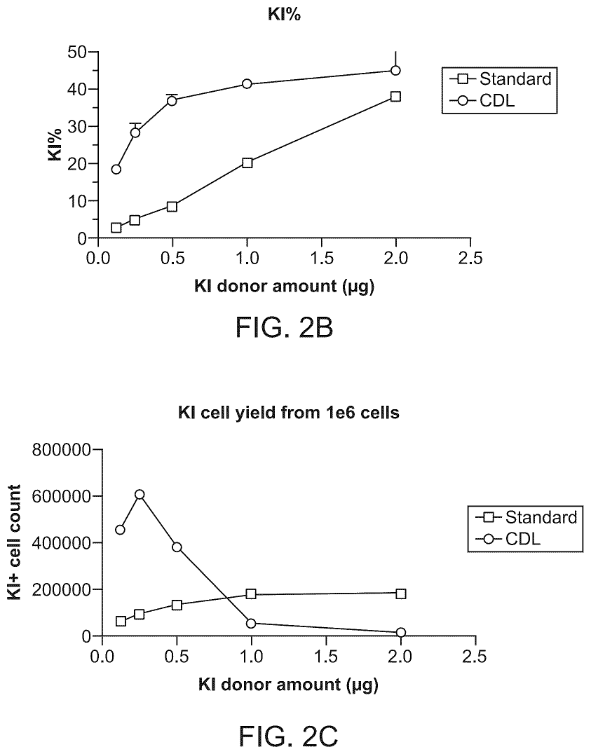

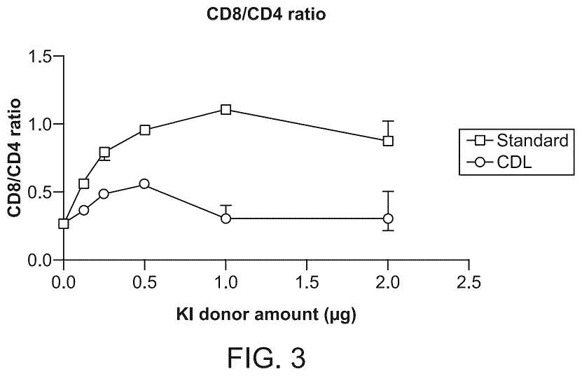

FIG. 2A-C are flow cytometry dot plots and graphs showing knock-in (KI) efficiency and KI cell yield of T-cells electroporated with a CRISPR RNP and homology-directed repair (HDR) templates of either standard plasmids or CDL plasmids. As shown in FIG. 2, T cells were electroporated with RNP complexes with varying amounts of either standard or CDL plasmid donor templates then assessed for transgene integration by flow cytometry (FIG. 2A, quantified in Table 1). The CDL design demonstrated greater KI % at lower doses of HDRT DNA than standard HDRT plasmids without the CDL sequence modifications (FIG. 2B, quantified in Table 2). Furthermore, overall T cell yield was increased at these low DNA doses compared to standard HDRT plasmids (FIG. 2C, quantified in Table 3). Accordingly, the CDL donor plasmid design demonstrated significantly (P<0,0001) increased yields of genetically engineered T cells compared to standard plasmid designs. Additionally, the ratio of CD8 to CD4 T cells was less than ˜0.5 when using the CDL HDRT template for all concentrations tested, in contrast to the standard HDRT plasmid (FIG. 3, quantified in Table 4). Variability of knock-in cell yield across T cells derived from 4 individual donors was also assessed by percent coefficient of variation (% CV) and maximal-to-minimal ratio (Max/Min fold), using either standard plasmids or CDL plasmids across varying doses. Notably, the difference between yields was distinctly lower with CDL designs as compared to standard plasmid designs at doses <=0.5 (Table 5). The data demonstrated the CDL donor plasmid design generated greater yield and greater consistency of yield across donors for electroporation-mediated Cas9 T cell editing and is a major benefit in the cell engineering process.

TABLE 1
Quantification of Knock-In Percentage
Representative Flow Images (Myc-Tag %)

| | | | | | | |
|---|---|---|---|---|---|---|
| KI % | Dose (ug) | 0.125 | 0.250 | 0.500 | 1.000 | 2.000 |

| | | | | | |
|---|---|---|---|---|---|
| Standard | 2.68 | 4.63 | 8.86 | 17.22 | 32.70 |
| CDL | 21.98 | 29.57 | 33.87 | 35.99 | 42.62 |

TABLE 2
Quantification of Knock-In Percentage (Myc-Tag %)

| KI % | Dose (ug) | 0.125 | 0.250 | 0.500 | 1.000 | 2.000 |
|---|---|---|---|---|---|---|
| Standard | median | 2.95 | 4.77 | 8.45 | 19.86 | 37.35 |
| CDL | median | 18.29 | 26.80 | 37.15 | 41.58 | 46.15 |

TABLE 3
Quantification of Knock-In Cell Yield

| KI yield/le6 cells | Dose (ug) | 0.125 | 0.250 | 0.500 | 1.000 | 2.000 |
|---|---|---|---|---|---|---|
| Standard | median | 61829.3 | 92116.2 | 133162.1 | 174020.3 | 183254.9 |
| CDL | median | 453577.2 | 604795.1 | 377600.0 | 50655.0 | 14933.3 |

TABLE 4
Quantification of CD8/CD4 Ratio

| CD8/CD4 ratio | Dose (ug) | 0.000 | 0.125 | 0.250 | 0.500 | 1.000 | 2.000 |
|---|---|---|---|---|---|---|---|
| Standard | median | 0.27 | 0.56 | 0.79 | 0.96 | 1.11 | 0.87 |
| CDL | median | 0.27 | 0.36 | 0.49 | 0.55 | 0.30 | 0.31 |

TABLE 5
Variability of knock-in cell yield across multiple donors

| | Standard | | CDL | |
|---|---|---|---|---|
| Dose (ug) | % CV | Max/Min fold | % CV | Max/Min fold |
| 0.125 | 80.36 | 5.69 | 41.58 | 2.21 |
| 0.250 | 83.75 | 8.02 | 52.84 | 3.62 |
| 0.500 | 92.34 | 13.40 | 89.28 | 22.56 |
| 1.000 | 99.75 | 33.37 | 119.03 | 144.78 |
| 2.000 | 101.37 | 45.00 | 147.25 | 162.58 |

Example 3—Performance on a Clinical Scale Platform and Inclusion of the CDL Sequence in the HDR Cassette Increases the Percentage of Cells with Transgene Integration Following Clinical Scale Transfection

### Materials and Methods

Isolation and activation of primary human T cells. Fresh cell populations collected by apheresis from healthy donors were obtained from HemaCare Corporation. CD4 and CD8 T cells were isolated from the donor cell population using the Miltenyi CD4 CD8 isolation positive isolation kit and the Miltenyi AutoMACS Pro Separator. Isolated CD4/8 T cells were activated following isolation using ThermoFisher Dynabeads Human T-Activator CD3/CD28. Cells were activated in Miltenyi TexMACS medium supplemented with 3% human AB serum (Gemini Bio), and IL7 and IL15 (Miltenyi Biotech). The cells were incubated for 48 hours at 37° C. following addition of activation medium.

Preparation of primary human T cells for electroporation: Following activation, the T cells were removed from the activation vessel and transferred to 50 mL conical tubes. The protocol provided by Miltenyi Biotech for bead removal was followed to obtain a bead-free, activated cell population.

Ribonucleoprotein complex preparation for electroporation: Single guide RNA targeting the GS94 locus was obtained from Synthego Corporation and resuspended to working concentration in TE/water. The caspase protein spCas9 was obtained from Aldevron. Guide RNA and caspase protein were complexed at room temperature to form the ribonucleoprotein (RNP) complex.

Plasmid DNA. Plasmid DNA containing the CDL sequences was designed in house and prepared by Elim Bio.

Electroporation mediated transfection of primary T cells. The activated T cells prepared as described above were counted and 5e7 cells per electroporation reaction were transferred to 50 ml conical tubes. The cells were pelleted at 300×g for 5 minutes then washed with DPBS and pelleted at 300×g for 5 minutes. The cells were then resuspended in the Lonza P3 buffer. The volume of the buffer was calculated so that the total volume of cell suspension following addition of RNPs and DNA would be 1 mL. To assess the performance across multiple DNA constructs, three CDL and three non-CDL constructs comprising transgene “X” were selected for testing. For each construct, 20 μg of DNA was used per transfection. The DNA was mixed with the RNP solution, then mixed thoroughly with the cell suspension. The cells, RNP, and DNA suspension was then transferred to the Lonza LV cartridge. The cells were electroporated on the Lonza LV device and allowed to recover for 10 minutes at room temperature following electroporation. The cell mixture was then transferred to a G-rex 100 (Wilson Wolf Manufacturing) containing 350 mL of media and incubated at 37° C.

Analysis of results by flow cytometry. The results were collected via flow cytometry on day 6 following transfection or day 8 following activation. To prepare the cells for analysis, the control wells were mixed to obtain an homogenous solution and counted using the Nexcelom K2 Cellometer with AOPI stain to differentiate live and dead cells. Using the obtained value, 2e5 cells were removed from each well and transferred to a 96-well V-bottom plate. The cells were pelleted then washed once in Stain Buffer (BD Biosciences) and pelleted at 400×g for 5 minutes. During the centrifugation, the staining solution was prepared. The flow panel includes the following: Myc PE (BioLegend) and Zombie L/D stain (BioLegend). The cells were resuspended in the staining solution and incubated at 4° C. for 30 minutes. Following incubation, Stain Buffer was added to the wells and the cells pelleted at 400×g for 5 minutes. The cells were then washed once with Stain Buffer and resuspended in Stain Buffer supplemented with counting beads (ThermoFisher). The flow analysis was performed on the ThermoFisher Attune cytometer with results analyzed using FlowJo software.

### Results

Performance on a clinical scale platform. The effect of modifying the nucleic acid sequence of a plasmid containing a homology directed repair template to include the CDL sequence immediately upstream of the 5′ HDR arm and downstream of the 3′ HDR arm was assessed in the context of the previously described CRISPR-Cas9 system on a platform and scale relevant to clinical manufacturing of a cell therapy product. The purpose of this study was to demonstrate the utility of the CDL sequence in a clinical context and highlight the applicability of the technology to manufacturing cell therapy products.

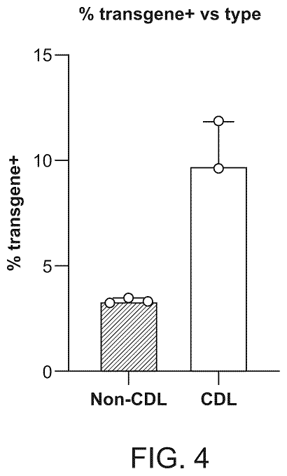

Inclusion of the CDL sequence in the HDR cassette increases the percentage of cells with transgene integration following clinical scale transfection. T cells electroporated with plasmids containing the CDL sequence GAGCCATGCTTGGCTTACGA (SEQ ID NO:2) and a sequence encoding a transgene or control plasmids comprising sequences encoding a transgene without the CDL sequence were assessed for both cell number and transgene expression on day 6 post-electroporation. The length of the polynucleotide sequence encoding the transgene was; 6533 base pairs in length (for plasmid pS3798), 7042 base pairs in length (for plasmid pS3631), and 6236 base pairs in length (for plasmid pS3797). The results shown in FIG. 4 indicate a significant (p<0.05) increase in transgene expression in the conditions receiving the plasmid containing the CDL sequence compared to the conditions receiving the control, non-CDL site containing plasmids. The difference is evident in the percentage of cells harboring an integrated transgene. On average, transgene expression as assessed by flow cytometry increased from 3.35% for the non-CDL conditions to 9.76% for the CDL conditions at 20 μg DNA. Overall, these data demonstrate the enhanced effect the inclusion of the CDL sequence has on the gene editing process.

### REFERENCES

1 Yin, H., Xue, W. & Anderson, D. G. CRISPR-Cas: a tool for cancer research and therapeutics. Nat Rev Clin Oncol, doi; 10.1038/s41571-019-0166-8 (2019).

2 Dunbar, C. E, et al. Gene therapy comes of age. Science 359, doi; 10.1126/science.aan4672 (2018).

3 Comu, T. I., Mussolino, C. & Cathomen, T. Refining strategies to translate genome editing to the clinic. Nat Med 23, 415-423, doi; 10.1038/nm.4313 (2017).

4 David, R. M. & Doherty, A. T. Viral Vectors: The Road to Reducing Genotoxicity. Toxicol Sci 155, 315-325, doi; 10.1093/toxsci/kfw220 (2017).

5 Roth, T. L, et al. Reprogramming human T cell function and specificity with non-viral genome targeting. Nature 559, 405-409, doi; 10.1038/s41586-018-0326-5 (2018).

6 Vo, L. T, et al. Regulation of embryonic haematopoietic multipotency by EZH1. Nature 553, 506-510, doi; 10.1038/nature25435 (2018).

7 Pouton, C. W., Wagstaff, K. M., Roth, D. M., Moseley, G. W. & Jans, D. A. Targeted delivery to the nucleus. Adv Drug Deliv Rev 59, 698-717, doi:10.1016/j.addr.2007.06.010 (2007).

8 Doudna, J. A. & Charpentier, E. Genome editing. The new frontier of genome engineering with CRISPR-Cas9. Science 346, 1258096, doi; 10.1126/science. 1258096 (2014).

10 Jiang, F. & Doudna, J. A. CRISPR-Cas9 Structures and Mechanisms. Annu Rev Biophys 46, 505-529, doi; 10.1146/annurev-biophys-062215-010822 (2017).

11 Luecke, S, et al, cGAS is activated by DNA in a length-dependent manner. EMBO Rep 18, 1707-1715, doi; 10.15252/embr.201744017 (2017).

12 Richardson, C. D., Ray, G. J., Bray, N. L. & Corn, J. E. Non-homologous DNA increases gene disruption efficiency by altering DNA repair outcomes. Nat Commun 7, 12463, doi; 10.1038/ncomms12463 (2016).

13 Bernkop-Schnurch, A. Strategies to overcome the polycation dilemma in drug delivery. Adv Drug Deliv Rev 136-137, 62-72, doi; 10.1016/j.addr.2018.07.017 (2018).

14 Vakulskas, C. A, et al. A high-fidelity Cas9 mutant delivered as a ribonucleoprotein complex enables efficient gene editing in human hematopoietic stem and progenitor cells. Nat Med 24, 1216-1224, doi; 10.1038/s41591-018-0137-0 (2018).

15 Ran, F. A, et al. Double nicking by RNA-guided CRISPR Cas9 for enhanced genome editing specificity. Cell 154, 1380-1389, doi; 10.1016/j.cell.2013.08.021 (2013).

1. A composition for modifying a target nucleic acid, comprising:
(a) a targetable nuclease protein; and
(b) a plasmid donor template comprising:
(i) a homology directed repair (HDR) template;
(ii) a first co-delivery linearization (CDL) target sequence, wherein the first CDL target sequence is operably linked 5′ of the HDR template; and
(iii) a second CDL target sequence, wherein the second CDL target sequence is operably linked 3′ of the HDR template, and
wherein each of the first and the second CDL target sequences are capable of cleavage by the targetable nuclease protein or a complex comprising the targetable nuclease protein, and
wherein the composition is formulated for non-viral delivery into a cell.

2. The composition of claim 1, wherein the targetable nuclease protein is an RNA-guided nuclease.

3. The composition of claim 2, wherein the composition further comprises an RNA comprising at least 17 nucleotides that are complementary to the first, second, or both first and second CDL target sequence.

4. The composition of claim 2, wherein the RNA-guided nuclease is a Cas protein.

5. The composition of claim 4, wherein the composition further comprises a donor guide RNA (gRNA) configured to form the complex comprising the targetable nuclease protein, and wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3′ of the CDL target sequences.

6. The composition of claim 4, wherein the composition further comprises a first donor guide RNA (gRNA) comprising at least 17 nucleotides that are complementary to the first CDL target sequence, a second donor gRNA comprising at least 17 nucleotides that are complementary to the second CDL target sequence, wherein each donor gRNA is configured to form a distinct complex comprising the targetable nuclease protein, and wherein each of the first and the second CDL target sequences are operably linked to a 3-base pair protospacer adjacent motif (PAM) located 3′ of the CDL target sequences.

7. The composition of claim 5, wherein one or more of the donor gRNAs comprises at least 17 nucleotides that are complementary to a genomic target sequence of the cell.

8. The composition of claim 7, wherein the HDR template comprises homology arms that are complementary to nucleic acid sequences flanking the genomic target sequence of the cell.

9. (canceled)

10. The composition of claim 1, wherein:
(a) the targetable nuclease comprises an RNA-guided nuclease, wherein the RNA-guided nuclease comprises CRISPR-CAS;
(b) the composition further comprises a donor guide RNA (gRNA) configured to form the complex comprising the targetable nuclease protein; and
(c) wherein (1) the donor gRNA comprises at least 17 nucleotides that are complementary to each of the first and the second CDL target sequences, and (2) each of the first and the second CDL target sequences are operably linked to a 3-base pair protospacer adjacent motif (PAM) located 3′ of the CDL target sequences.

11. (canceled)

12. The composition of claim 1, wherein the composition further comprises a second targetable nuclease protein, wherein the second targetable nuclease protein or a complex comprising the second targetable nuclease protein is capable of cleaving a genomic target sequence of the cell.

13. The composition of claim 12, wherein the second targetable nuclease protein is an RNA-guided nuclease.

14. The composition of claim 13, wherein the composition further comprises a second RNA comprising at least 17 nucleotides that are complementary to the genomic target sequence.

15. The composition of claim 13, wherein the RNA-guided nuclease is a Cas protein.

16. The composition of claim 15, wherein the composition further comprises a target guide RNA (gRNA) configured to form the complex comprising the second targetable nuclease protein, and wherein (1) the target gRNA comprises at least 17 nucleotides that are complementary to the genomic target sequence, and (2) the genomic target sequence is operably linked to a 3-base pair protospacer adjacent motif (PAM) located 3′ of the genomic target sequence.

17. (canceled)

18. The composition of claim 12, wherein the genomic target sequence comprises a safe-harbor nucleic acid sequence.

19. The composition of claim 18, wherein the safe-harbor nucleic acid sequence comprises the nucleic acid sequence GAGCCATGCTTGGCTTACGA.

20. The composition of claim 5, wherein one, both, or neither of the PAM sequences are encoded between the CDL target sequences and the HDR template.

21.-25. (canceled)

26. The composition of claim 1, wherein the targetable nuclease protein and/or the second targetable nuclease protein is a Cas9 protein, a transcription activator-like (TAL) effector DNA-binding protein and a nuclease, or a zinc finger DNA-binding protein and a nuclease.

27.-31. (canceled)

32. A ribonucleoprotein complex for modifying a target nucleic acid,
(a) a CRISPR-CAS RNA-guided nuclease; and
(b) a donor guide RNA (gRNA), wherein the donor gRNA comprises at least 17 nucleotides that are complementary to a co-delivery linearization (CDL) target sequence, and
wherein the composition is formulated for non-viral delivery into a cell.

33.-47. (canceled)

48. A method for modifying a target nucleic acid of a cell, the method comprising:
providing the cell, and
introducing or having introduced into the cell a composition formulated for non-viral delivery, the composition comprising:
(a) a targetable nuclease protein; and
(b) a plasmid donor template comprising:
(i) a homology directed repair (HDR) template;
(ii) a first co-delivery linearization (CDL) target sequence, wherein the first CDL target sequence is operably linked 5′ of the HDR template; and
(iii) a second CDL target sequence, wherein the second CDL target sequence is operably linked 3′ of the HDR template, and
wherein each of the first and the second CDL target sequences are capable of cleavage by the targetable nuclease protein or a complex comprising the targetable nuclease protein.

49.-60. (canceled)
